(19) United States
(12) Patent Application Publication
(10) Pub. No.: US 2010/0120839 A1
(43) Pub. Date: May 13, 2010

(54) PYRAZOLES USEFUL IN THE TREATMENT OF INFLAMMATION

(75) Inventors: Kiyo No, Uppsala (SE); Andrei Sanin, Uppsala (SE); Gasse Kromann, Copenhagen (DK)

Correspondence Address: K&L Gates LLP, 1900 MAIN STREET, SUITE 600, IRVINE, CA, 92614-7319, US

(73) Assignee: Biolipox AB

(21) Appl. No.: 12/596,652

(22) PCT Filed: Apr. 21, 2008
(86) PCT No.: PCT/GB08/01387

§ 371 Date: Dec. 31, 2009

Related U.S. Application Data
(60) Provisional application No. 60/907,885, filed on Apr. 20, 2007.

(51) Int. Cl. A61K 31/437; C07D 263/54; A61K 31/423; C07D 498/04; A61P 29/00; A61P 11/06; A61P 1/00; A61P 37/08; A61P 37/06; A61P 9/00; A61P 25/28

(52) U.S. Cl. 514/302; 548/217; 514/375; 546/115

(57) ABSTRACT

There is provided compounds of formula I,

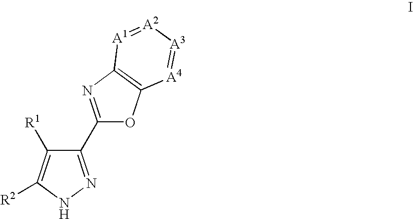

wherein R¹, R², A¹, A², A³ and A⁴ have meanings given in the description, and pharmaceutically-acceptable salts thereof, which compounds are useful in the treatment of diseases in which inhibition of the activity of a lipoxygenase (e.g. 15-lipoxygenase) is desired and/or required, and particularly in the treatment of inflammation.

### FIELD OF THE INVENTION

The invention relates to novel pharmaceutically-useful compounds. The invention further relates to compounds that are useful in the inhibition of the activity of 15-lipoxygenase and thus in the treatment of inflammatory diseases and of inflammation generally. The invention also relates to the use of such compounds as medicaments, to pharmaceutical compositions containing them, and to synthetic routes for their production.

### BACKGROUND OF THE INVENTION

There are many diseases/disorders that are inflammatory in their nature. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, leukotriene receptor antagonists (LTRas) have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, a new and/or alternative anti-inflammatory treatment would be of benefit to all of the above-mentioned patient groups. In particular, there is a real and substantial unmet clinical need for an effective anti-inflammatory drug capable of treating inflammatory disorders, such as asthma, with no real or perceived side effects.

The mammalian lipoxygenases are a family of structurally-related enzymes, which catalyze the oxygenation of inter alia arachidonic acid. Three types of human lipoxygenases are known, which catalyze the insertion of molecular oxygen into arachidonic acid at carbon positions 5, 12 and 15. The enzymes are thus named 5-, 12- and 15-lipoxygenase, respectively.

Arachidonic acid metabolites that are formed following the action of lipoxygenases are known to have pronounced pathophysiological activity including pro-inflammatory effects.

For example, the primary product of the action of 5-lipoxygenase on arachidonic acid is further converted by a number of enzymes to a variety of physiologically and pathophysiologically important metabolites. The most important of these, the leukotrienes, are strong bronchoconstrictors. Huge efforts have been devoted towards the development of drugs that inhibit the action of these metabolites as well as the biological processes that form them. Drugs that have been developed to this end include 5-lipoxygenase inhibitors, inhibitors of FLAP (Five Lipoxygenase Activating Protein) and, as mentioned previously, leukotriene receptor antagonists (LTRas).

Another class of enzymes that metabolize arachidonic acid are the cyclooxygenases. Arachidonic acid metabolites that are produced by this process include prostaglandins, thromboxanes and prostacyclin, all of which possess physiological or pathophysiological activity. In particular, the prostaglandin PGE₂ is a strong pro-inflammatory mediator, which also induces fever and pain. Consequently, a number of drugs have been developed to inhibit the formation of PGE₂, including “NSAIDs” (non-steroidal antiinflammatory drugs) and “coxibs” (selective cyclooxygenase-2 inhibitors). These classes of compounds act predominantly by way of inhibition of one or several cyclooxygenases.

Thus, in general, agents that are capable of blocking the formation of arachidonic acid metabolites are likely to be of benefit in the treatment of inflammation.

The listing or discussion of an apparently prior-published document in this specification should not necessarily be taken as an acknowledgement that the document is part of the state of the art or is common general knowledge.

A 2-pyrazolobenzoxazole has been disclosed as a potential antimicrobial drug in Vinsova et al, Bioorganic & Medicinal Chemistry (2006), 14(17), 5850-5865. However, there is no mention in that document of the use of any of the compounds disclosed therein as inhibitors of lipoxygenases, and therefore in the treatment of inflammation.

Certain pyrazolecarboxylic acid hydrazides, structurally unrelated to the compounds described herein, have been disclosed as anti-inflammatory agents in Tihanyi et al, Eur. J. Med. Chem.—Chim. Ther., 1984, 19, 433 and Goel et al, J. Chem. Inf. Comput. Sci. 1995, 35, 510.

Vertuani et al., Journal of Pharmaceutical Sciences, Vol. 74, No. 9 (1985) discloses various pyrazoles that possess anti-inflammatory and analgesic activities. There is no mention or suggestion of pyrazoles that are substituted at the 3-position with an oxazole ring.

U.S. Pat. No. 6,166,041 discloses various benzoxazole PDE IV inhibitors for the treatment of asthma. However, this document only discloses benzoxazoles that are substituted, either directly attached or via a linker group, at the benzene ring of the benzoxazole (at the 7-position) with a phenyl ring or monocyclic ring containing at least one heteroatom.

International patent application WO 2006/092430 and national patent application DE 10 2005 009 705 disclose various bicyclic compounds, that are primarily naphthalenes, that may be useful in the inhibition of human corticosteroid synthase, and therefore in the treatment of diseases such as hypercortisolism and diabetes mellitus. However, there is no mention of the use of the compounds disclosed therein as lipoxygenase inhibitors that are therefore useful in the treatment of inflammation.

International patent application WO 2007/019417 discloses various compounds as sirtuin modulators. However, that document only discloses compounds comprising a series of three aromatic groups, and, further, there is no disclosure therein of the use of such compounds as inhibitors of lipoxygenases that are therefore useful in the treatment of inflammation.

International patent application WP 2006/04614 discloses compounds containing aromatic groups, which compounds may be useful as glucocorticoid receptor modulators. Such compounds include inter alia pyrazoles. However, this document does not disclose pyrazoles that are not substituted with a sulfonamide group.

International patent applications WO 2004/080999 and WO 2006/032852 both disclose various 3-amidopyrazoles for use in the treatment of inflammation. However, there is no disclosure or suggestion in either of these documents of N-unsubstituted pyrazoles for use in such treatment.

US patent application US 2005/0070589 discloses various pyrazoles as potential inhibitors of 15-lipoxygenase. However, there the pyrazoles disclosed therein are necessarily substituted at the 4-position with an alkyl group that is necessarily substituted with a substituent containing a nitrogen heteroatom. US patent application US 2004/0198768 discloses various bicyclic compounds, including benzazoles, that may be useful as 5-lipoxygenase inhibitors. However, such benzazoles are necessarily substituted at the 2-position with an aminophenyl group. European patent application EP 0 418 845 and international patent application WO 2004/080999 both disclose various pyrazoles for use as medicaments. However, both documents only mention pyrazoles that are 1(N)-substituted.

International patent application WO 2006/032851 discloses various 3-amidopyrazoles for use in the treatment of inflammation. Further, international patent applications WO 2007/045868, WO 2007/051981, WO 2007/052000 and WO 2007/051982 disclose either 3-amidopyrazoles or amidotriazoles for use in the treatment of inflammation. However, there is no disclosure or suggestion in any of these documents of pyrazoles that are substituted in the 3-position with an oxazole group.

### DISCLOSURE OF THE INVENTION

According to the invention there is provided a compound of formula I,

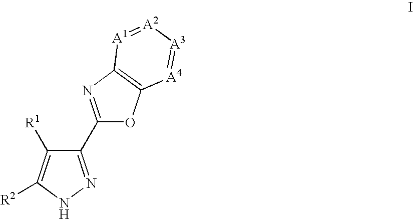

wherein,
R¹ and R² independently represent H, halo, C₁₋₆ alkyl or —O—C₁₋₆ alkyl, which latter two groups are optionally substituted by one or more halo atoms;
A¹, A², A³ and A⁴ each respectively represent —C(R³)═, —C(R⁴)═, —C(R⁵)═ and —C(R⁶)═, or, each of these represent —N═;
R³, R⁴, R⁵ and R⁶ independently represent hydrogen or X¹;
X¹ represents halo, —R^{3a}, —CN, —C(O)R^{3b}, —C(O)OR³ⁿ, —C(O)N(R^{4a})R^{5a}, —N(R^{4b})R^{5b}, —N(R^{3d})C(O)R⁴ⁿ, —N(R^{3e})C(O)N(R^{4d})R^{5d}, —N (R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³¹, —S(O)ₘR^{3j}, —N(R^{3k})S(O)₂R^{3m}, —OC(O)R³ⁿ, —OC(O)OR³P, —S(O)₂N(R^{4b})R^{5b}, —S(O)₂OH, —P(O)(OR⁴ⁱ)(OR⁵ⁱ) or —C(O)N(R^{3q})S(O)₂R^{3r};
m represents 0, 1 or 2;
R^{3a} represents C₁₋₆ alkyl optionally substituted by one or more substituents selected from halo (e.g. F or Cl), —N(R^{6a})R^{6b}, —N₃, ═O and —OR^{6c};
R^{3b} to R^{3h}, R^{3k}, R³ⁿ, R^{3q}, R^{4a} to R^{4h}, R^{5a}, R^{5b}, R^{5d} and R^{5f} to R^{5h} independently represent H or C₁₋₆ alkyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h}, may be linked together to form a 3- to 6-membered ring, which ring optionally contains a further heteroatom (such as nitrogen or oxygen) in addition to the nitrogen atom to which these substituents are necessarily attached, and which ring is optionally substituted by F, ═O and/or C₁₋₆ alkyl optionally substituted by one or more fluoro atoms;
R³ⁱ, R^{3j}, R^{3m}, R^{3p} and R^{3r} independently represent C₁₋₆ alkyl optionally substituted by one or more substituents selected from B¹;
R⁴ⁱ and R⁵ⁱ independently represent H or C₁₋₆ alkyl optionally substituted by one or more substituents selected from B²;
R^{6a}, R^{6b}, R^{6c} and R^{6d} independently represent H or C₁₋₆ alkyl optionally substituted by one or more substituents selected from B³; or
R^{6a} and R^{6b} may be linked together to form a 3- to 6-membered ring, which ring optionally contains a further heteroatom (such as nitrogen or oxygen) in addition to the nitrogen atom to which these substituents are necessarily attached, and which ring is optionally substituted by ═O and/or C₁₋₆ alkyl optionally substituted by one or more fluoro atoms;
B¹, B² and B³ independently represent F, Cl, —OCH₃, —OCH₂CH₃, —OCHF₂, —OCH₂CF₃, —OCF₃ or —OCF₂CF₃,
or a pharmaceutically-acceptable salt thereof,
provided that:
when R¹ and R² both represent H, A¹, A², A³ and A⁴ respectively represent —C(R³)═, —C(R⁴)═, —C(R⁵)═ and —C(R⁶)═, and R³ and R⁵ represent H, then both R⁴ and R⁶ do not represent t-butyl,
which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group). Further, when there is a sufficient number (i.e. a minimum of four) of carbon atoms, such groups may also be part cyclic. Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

The term “halo”, when used herein, includes fluoro, chloro, bromo and iodo.

Aryl groups that may be mentioned include C₆₋₁₄ (e.g. C₆₋₁₀) aryl groups. Such groups may be monocyclic, bicyclic or tricyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. Heteroaryl groups that may be mentioned include those which have between 5 and 14 (e.g. between 5 and 10) members. Such groups may be monocyclic, bicyclic or tricyclic, provided that at least one of the rings is aromatic and wherein at least one (e.g. one to four) of the atoms in the ring system is other than carbon (i.e. a heteroatom).

Heteroatoms that may be mentioned include phosphorus, silicon, boron, tellurium, selenium and, preferably, oxygen, nitrogen and sulphur.

For the avoidance of doubt, when a phrase such as “R^{3b} to R^{3h}” is employed herein, this will be understood by the skilled person to mean R^{3b}, R^{3c}, R^{3d}, R^{3e}, R^{3f}, R^{3g} and R^{3h} inclusively.

For the avoidance of doubt, where it is stated that A¹, A², A³ and A⁴ each respectively represent —C(R³)═, —C(R⁴)═, —C(R⁵)═ and —C(R⁶)═, or, each of these represent —N═, we mean that: A¹ may represent —C(R³)═ or —N═; A² may represent —C(R⁴)═ or —N═; A³ may represent —C(R⁵)═ or —N═; and A⁴ may represent —C(R⁶)═ or —N═. That is, A¹, A², A³ and A⁴ respectively represent —C(R³)═, —C(R⁴)═, —C(R⁵)═ and —C(R⁶)═, or, each of these may alternatively and independently represent —N═.

For the avoidance of doubt, in cases in which the identity of two or more substituents in a compound of formula I may be the same, the actual identities of the respective substituents are not in any way interdependent. For example, in the situation in which two or more of R³, R⁴, R⁵ and R⁶ are present and represent X¹, in which the two (or more) X¹ groups are R^{3a}, in which R^{3a} is a C₁₋₆ alkyl group, the respective alkyl groups may be the same or different. Hence, where there is, for example, more than one X¹ group present in the compound of formula I, then such groups are not in any way interdependent, i.e. they may be the same or different.

Compounds of the invention that may be mentioned include those in which:

R^{3b} to R^{3h}, R^{3k}, R³ⁿ, R^{3q}, R^{4a} to R^{4h}, R^{5a}, R^{5b}, R^{5d} and R^{5f} to R^{5h} independently represent H or C₁₋₆ alkyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h}, may be linked together to form a 3- to 6-membered ring, which ring optionally contains a further heteroatom (such as nitrogen or oxygen) in addition to the nitrogen atom to which these substituents are necessarily attached, and which ring is optionally substituted by ═O and/or C₁₋₆ alkyl optionally substituted by one or more fluoro atoms.

Preferred compounds of the invention that may be mentioned include those in which R¹ and R² (in particular R¹) independently represent —O—C₁₋₆ alkyl (optionally substituted by one or more halo atoms) or, more preferably, H or halo.

Preferred compounds of the invention include those in which R¹ and R² independently represent H, halo or C₁₋₆ (e.g. C₁₋₃) alkyl optionally substituted by one or more halo (e.g. fluoro) atoms.

Preferred compounds of the invention also include those in which:

when R¹ and R² represent halo, then it is preferably fluoro or chloro;
when R¹ and R² represent optionally substituted C₁₋₆ alkyl or —O—C₁₋₆ alkyl, then they are preferably, C₁₋₃ alkyl or —O—C₁₋₃ alkyl (both of which are) optionally substituted by one or more halo atoms;
R¹ and R² independently represent C₁₋₃ (e.g. C₁₋₂) alkyl (which alkyl group is optionally substituted as hereinbefore defined, for example by one or more halo (e.g. fluoro) atoms) or, more preferably, H or halo (e.g. F or Cl); any three, preferably any two, or, more preferably any one of A¹ to A⁴ represents —N═ and the others represent (as appropriate) —C(R³)═, —C(R⁴)═, —C(R⁵)═ or —C(R⁶)═;
m represents 2;
when any of the pairs R^{4a} and R^{5a}, R^{4b} and R^{5b}, R^{4d} and R^{5d}, R^{4f} and R^{5f}, R^{4g} and R^{5g}, R^{4h} and R^{5h}, and R^{6a} and R^{6b} are linked together, they form a 5- to 6-membered ring, which ring optionally contains a further heteroatom (such as nitrogen or oxygen) and is optionally substituted by methyl, —CHF₂ or CF₃ (so forming, for example, a pyrrolidinyl, piperidinyl, morpholinyl or a piperazinyl (e.g. 4-methylpiperazinyl) ring);
R^{3a} represents C₁₋₆ alkyl optionally substituted by one or more substituents selected from halo (e.g. fluoro) and —OR^{6c};
R^{3b} to R^{3h}, R^{3k}, R³ⁿ, R^{3q}, R^{4a} to R^{4h}, R^{5a}, R^{5b}, R^{5d} and R^{5f} to R^{5h} independently represent H or C₁₋₄ (e.g. C₁₋₃) alkyl optionally substituted by one or more halo atoms or —OR^{6d};
R³ⁱ, R^{3j}, R^{3m}, R^{3p} and R^{3r} independently represent C₁₋₄ (e.g. C₁₋₃) alkyl optionally substituted by one or more substituents selected from B¹;
R⁴ⁱ and R⁵ⁱ independently represent H or C₁₋₄ (e.g. C₁₋₃) alkyl optionally substituted by one or more substituents selected from B²;
R^{6a}, R^{6b}, R^{6c} and R^{6d} independently represent H or C₁₋₃ alkyl optionally substituted by one or more substituents selected from B³;
B¹, B² and B³ independently represent —OCF₃, —OCH₃ or, more preferably, F or Cl.

Further preferred compounds of the invention include those in which:

X¹ represents halo (e.g. F, Cl or Br), —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j} or —S(O)₂N(R^{4h})R^{5b};
R^{3a} represents C₁₋₄ alkyl (e.g. ethyl, isopropyl, t-butyl, cyclopropyl, cyclobutyl, cyclopropylmethyl or, especially, methyl) optionally substituted by one or more halo (e.g. fluoro) atoms (so forming, for example, a —CHF₂ or CF₃ group);
R^{3b}, R^{3c}, R^{3h}, R^{4a} to R^{4h}, R^{5a}, R^{5b}, R^{5d} and R^{5f} to R^{5h} independently represent hydrogen or C₁₋₄ (e.g. C₁₋₃) alkyl (e.g. methyl), or the relevant pairs (i.e. R^{4a} and R^{5a}, R^{4b} and R^{5b}, R^{4d} and R^{5d}, R^{4f} and R^{5f}, R^{4g} and R^{4g} and R^{4h} and R^{5h}) are linked together as hereinbefore defined;
R^{3d} to R^{39g} independently represent C₁₋₂ alkyl (e.g. methyl) or, more particularly, hydrogen;
R³ⁱ and R^{3j} independently represent C₁₋₄ (e.g. C₁₋₂) alkyl (e.g. methyl) optionally substituted by one or more F atoms (so forming, for example a CF₃ group).

More preferred compounds of formula I include those in which:

X¹ represents —C(O)N(R^{4a})R^{5a}, —N(R^{4b})R^{5b}, —N(H)C(O)R^{4c}, —S(O)₂CH₃, —S(O)₂CF₃, —S(O)₂N(R^{4h})R^{5b}, preferably, —CN or —NO₂, or, more preferably halo, —R^{3a} or —OR^{3b};
R^{3h} represents hydrogen or C₁₋₄ (e.g. C₁₋₃) alkyl (e.g. ethyl, isopropyl, t-butyl, cyclopropyl, cyclobutyl, cyclopropylmethyl or, more preferably, methyl) optionally substituted by one or more fluoro atoms (so forming, for example, —CHF₂ or CF₃);
the pairs R^{4a} and R^{5a}, R^{4b} and R^{5b} and R^{4h} and R^{5h} are linked together to form a pyrrolidinyl, piperidinyl, morpholinyl or a piperazinyl (e.g. 4-methylpiperazinyl) ring or, more preferably, R^{4a}, R^{4b}, R^{4c}, R^{4h}, R^{5a}, R^{5b} and R^{5h} independently represent hydrogen, methyl or ethyl.

Preferred compounds of the invention include those in which:

X¹ represents halo (e.g. chloro or fluoro), R^{3a} or —OR^{3h};
when any one of A¹ to A⁴ (e.g. A¹ or A⁴) represents —N═, then when at least one (e.g. one) X¹ substituent is present, it (or at least one) is preferably located at the R⁵ or particularly the R⁴ position;
R^{3a} represents C₁₋₃ alkyl (e.g. methyl) optionally substituted by one or more halo (e.g. fluoro) atoms (so forming, for example, a difluoromethyl or preferably a trifluoromethyl group);
R^{3h} represents C₁₋₃ alkyl (optionally substituted by one or more halo (e.g. fluoro) atoms) or, more preferably, H;
R³, R⁴, R⁵ and R⁶ independently represent trifluoromethyl or, preferably, H, methyl, fluoro, chloro or hydroxy.

More preferred compounds of the invention include those in which:

R¹ and R² independently represent methyl (optionally substituted by one or more fluoro atoms; so forming for example a trifluoromethyl or, preferably a difluoromethyl group) or, more preferably, H or Cl;
when one of R¹ and R² represents a substituent other than hydrogen, then it is preferably R²;
any one of A¹ to A⁴ represents —N═ or none of A¹ to A⁴ represent —N═ (and the others, as appropriate/applicable, represent —C(R³)═, —C(R⁴)═, —C(R⁵)═ or —C(R⁶)═);
when any one of A¹ to A⁴ represent —N═, then it is preferably A¹ or A⁴ that represents that group;
A¹ represents —C(R³)═ or —N═;
R³ represents H, methyl or fluoro;
A² represents —C(R⁴)═;
R⁴ represents methyl (optionally substituted by one or more fluoro atoms, so forming for example a CHF₂ or, preferably a CF₃ group) or, preferably, H, chloro or fluoro;
A³ represents —C(R⁵)═;
R⁵ represents H, chloro, fluoro, methyl or hydroxy;
A⁴ represents —N═ or, more preferably, —C(R⁶)═;
R⁶ represents H, chloro or fluoro.

Preferred substituents on the A¹ to A⁴ containing ring include trifluoromethyl and, preferably, chloro, fluoro, methyl and/or hydroxy substituents.

Particularly preferred compounds of formula I include those of the examples described hereinafter.

Compounds of formula I may be made in accordance with techniques that are well known to those skilled in the art, for example as described hereinafter.

According to a further aspect of the invention there is provided a process for the preparation of a compound of formula I, which process comprises:

(i) for compounds of formula I in which R² represents halo or C₁₋₆ alkyl (optionally substituted by one or more halo atoms), reaction of a corresponding compound of formula I in which R² represents hydrogen, with an appropriate base (or a mixture of bases), such as potassium bis(trimethylsilyl)amide, sodium bis(trimethylsilyl)amide, sodium hydride, potassium tert-butoxide or an organolithium base, such as n-BuLi, s-BuLi, t-BuLi, lithium diisopropylamide or lithium 2,2,6,6-tetramethylpiperidine (which organolithium base is optionally in the presence of an additive (for example, a lithium co-ordinating agent such as an ether (e.g. dimethoxyethane) or an amine (e.g. tetramethylethylenediamine (TMEDA), (−)sparteine or 1,3-dimethyl-3,4,5,6-tetrahydro-2(1H)-pyrimidinone (DMPU) and the like)) followed by quenching with an appropriate electrophile such as:
(a) for compounds of formula I in which R² represents an optionally substituted C₁₋₆ alkyl group, a compound of formula II,

R^{c}L^{1a} II
wherein R^{c} represents C₁₋₆ alkyl (which alkyl group is optionally substituted by one or more halo atoms), and L^{1a} represents a suitable leaving group such as halo (e.g. iodo or bromo) or a sulfonate group (such as —OSO₂CF₃, OSO₂CH₃ and —OSO₂-aryl (e.g. —O-tosyl)) or, for compounds of formula I in which R² represents CF₃, a trifluoromethylating reagent, such as 5-(trifluoromethyl)-dibenzothiophenium tetrafluoroborate; or
(b) for compounds of formula I in which R² represents halo, an electrophile that provides a source of these atoms. For example, for bromine atoms, reagents include N-bromosuccinimide, bromine and 1,2-dibromotetrachloroethane, for chlorine atoms reagents include N-chlorosuccinimide, chlorine, iodine monochloride and hexachloroethane, for iodine atoms, appropriate reagents include iodine, 1,2-diiodoethane and 1,2-diiodotetrachloroethane and for fluorine atoms reagents include xenon difluoride, SELECTFLUOR® ([1-(chloromethyl)-4-fluoro-1,4-diazonia-bicyclo[2.2.2]octane bis(tetrafluoroborate)]), CF₃OF, perchloryl fluoride, F₂ and acetylhypofluoride.

The skilled person will appreciate that the corresponding compounds of formula I in which R² represents hydrogen (on which the above reaction is performed) are preferably protected at the nitrogen atom of the pyrazole ring system, preferably with a protective group that is also a directing metallation group (such as a benzenesulfonyl group or a SEM (i.e. a —CH₂OC₂H₄Si(CH₃)₃) group). The reaction may be performed in the presence of a suitable solvent, such as a polar aprotic solvent (e.g. tetrahydrofuran or diethyl ether), at sub-ambient temperatures (e.g. 0° C. to −78° C.) under an inert atmosphere followed (as appropriate) by deprotection of the N-protective group under standard conditions (e.g. when a benzenesulfonyl group is employed, by hydrolysis or, when a SEM group is employed by reaction in the presence of CsF, LiBF₄, Bu₄NF, HF/pyridine, MgBr₂/BuSH or in the presence of acids (such as HCl in methanol or ethanol and TFA in dichloromethane at rt or even stronger acidic conditions such as HBr/AcOH or HCl/H₂O for instance at elevated temperature);

(ii) for compounds of formula I in which R¹ and/or R² represent C₁₋₆ alkoxy (optionally substituted by one or more halo atoms), reaction of a compound corresponding to a compound of formula I but in which in place of the relevant substituents R¹ and/or R² (as appropriate), (a) hydroxy group(s) is/are present, with a compound of formula II as hereinbefore defined, in which R^{c} represents C₁₋₆ alkyl (optionally substituted by one or more halo substituents), or (for the introduction of a methoxy group at R¹ and/or R²) with diazomethane or trimethylsilyldiazomethane. Each reaction may be performed under standard conditions known to those skilled in the art, for example the former may be performed in the presence of base (e.g. sodium hydride) and a suitable solvent (e.g. dimethylformamide or tetrahydrofuran) and the latter may be performed in the presence of a suitable solvent (e.g. an aromatic hydrocarbon such as benzene or a di(alkyl)ether such as diethyl ether). In the case of the latter, diazomethane may be prepared from Diazald®;
(iii) for compounds of formula I in which R² represents CF₃, reaction of a corresponding compound of formula I in which R² represents bromo or, preferably, iodo with CuCF₃ (or a source of CuCF₃) in, for example, the presence of HMPA and DMF. The skilled person will appreciate that the reagent CuCF₃ may not be isolated as such, and may be prepared in accordance with the procedures described in Burton D. G.; Wiemers D. M.; J. Am. Chem. Soc., 1985, 107, 5014-5015 and Mawson S. D.; Weavers R. T.; Tetrahedron Letters., 1993, Vol. 34, No. 19, 3139-3140 (for example, by the reaction of zinc and e.g. CF₂Br₂ in DMF so forming ZnCF₃ (or a source thereof) followed by treatment with CuBr in HMPA);
(iv) reaction of a compound of formula III,

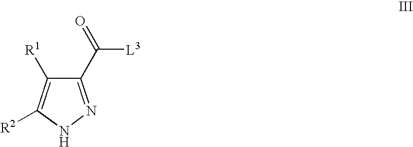

or a protected (e.g. N-protected) derivative thereof, wherein R¹ and R² are as hereinbefore defined, and L³ represents a suitable leaving group, such as chloro, bromo, or a hydroxy group, which latter group may, if necessary be activated by treatment with a suitable reagent (e.g. oxalyl chloride, thionyl chloride, etc) optionally in the presence of an appropriate solvent (e.g. dichloromethane, THF, toluene or benzene) and a suitable catalyst (e.g. DMF), resulting in the formation of the respective acyl chloride, with a compound of formula IV,

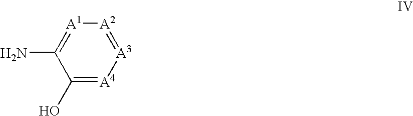

wherein A¹, A², A³ and A⁴ are as hereinbefore defined, under standard condensation/dehydration conditions, for example, in the presence of a reagent (e.g. an acid) that effects the cyclisation. Such conditions include reaction in the presence of a reagent such as P₂O₅ or an acid (such as polyphosphoric acid) at room or, preferably, elevated temperature (e.g. at reflux). The reaction is preferably performed on compounds of formula III in which L³ represents hydroxy (without activation to the corresponding acyl chloride) with compounds of formula IV, in the presence of polyphosphoric acid, for example at elevated temperature (e.g. about 160° C.);
(v) intramolecular cyclisation of a compound of formula V,

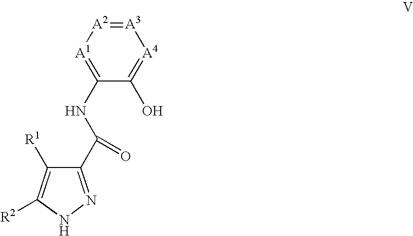

or a compound of formula VI,

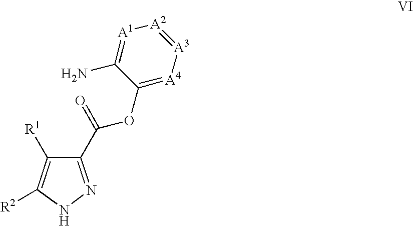

wherein R¹, R², A¹, A², A³ and A⁴ are as hereinbefore defined, for example under conditions such as those described hereinbefore in respect of preparation of compounds of formula I (process step (iv) above);
(vi) for compounds of formula I in which R² represents hydrogen and R¹ is as hereinbefore defined, removal of the group J from a compound of formula VII,

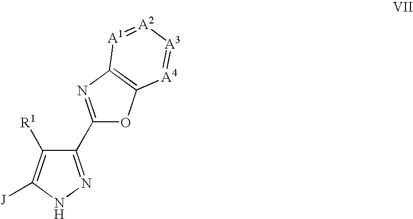

wherein J represents —Si(R^{t})₃ or —Sn(R^{z})₃ (in which each R^{t} independently represents a C₁₋₆ alkyl (e.g. a methyl or isopropyl) group or an aryl (e.g. phenyl) group and each R^{z} independently represents C₁₋₆ alkyl (e.g. methyl or butyl)), and R¹, A¹, A², A³ and A⁴ are as hereinbefore defined. When J represents —Si(R^{t})₃, the reaction may be performed in the presence of an appropriate reagent for the removal of the silyl group, such as a source of halide anions (e.g. tetrabutylammonium fluoride, tetramethylammonium fluoride, hydrogen fluoride or potassium fluoride), for example, in the presence of a suitable solvent (e.g. tetrahydrofuran) at room temperature. When J represents —Sn(R^{z})₃, the reaction may be a standard hydrolysis, for example reaction with water or an aqueous acid (e.g. hydrochloric acid) in the presence of an appropriate solvent (e.g. dioxane, tetrahydrofuran, MeOH or EtOH (or mixtures thereof));
(vii) for compounds of formula I in which one of R¹ or R² represents an optionally substituted C₁₋₆ alkyl group, chloro or fluoro and the other represents H, reaction of a corresponding compound of formula I in which one of R¹ or R² represents bromo or iodo and the other represents H (as appropriate) with a suitable organolithium base (e.g. t-BuLi, s-BuLi or n-BuLi) optionally in the presence of an additive (such as one hereinbefore described in respect of process step (i)), followed by quenching with a compound of formula II, as hereinbefore defined, or a source of chlorine or fluorine atoms, such as one described in respect of process (i) above. This reaction may be performed in the presence of a suitable solvent, such as one hereinbefore described in respect of process step (i) at low temperatures (e.g. −78 to −120° C.) under an inert atmosphere;
(viii) for compounds of formula I in which R² represent H or C₁₋₆ alkyl optionally substituted by one or more halo atoms, reaction of a compound of formula VIIA,

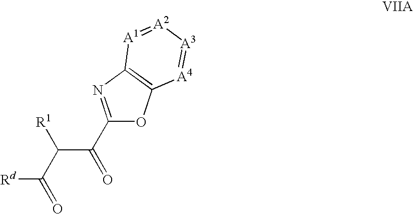

wherein R^{d} represents H or C₁₋₆ alkyl optionally substituted by one or more halo atoms and R¹ is as hereinbefore defined, with hydrazine (or a hydrate or derivative (e.g. benzylhydrazine) thereof), for example under condensation reaction conditions in the presence of an alcoholic solvent (e.g. ethanol) at elevated temperature (e.g. at reflux);
(ix) reaction of a compound of formula VIIB,

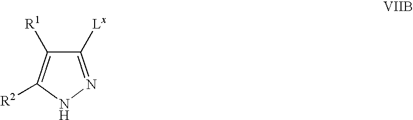

wherein L^{x} represents a suitable leaving group such as chloro, bromo, iodo, a sulfonate group (e.g. —OS(O)₂CF₃, —OS(O)₂CH₃, —OS(O)₂PhMe or a nonaflate), —B(OH)₂, —B(OR^{wx})₂, —Sn(R^{wx})₃ or diazonium salts, in which each R^{wx} independently represents a C₁₋₆ alkyl group, or, in the case of —B(OR^{wx})₂, the respective R^{wx} groups may be linked together to form a 4- to 6-membered cyclic group (such as a 4,4,5,5-tetramethyl-1,3,2-dioxaborolan-2-yl group), and R¹ and R² are as hereinbefore defined, with a compound of formula VIIC,

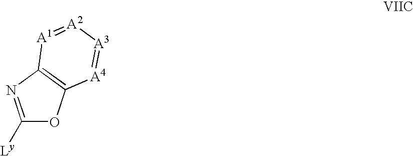

wherein L^{y} represents a suitable leaving group, such as one hereinbefore defined in respect of Lx, and, in particular represents chloro, bromo, iodo, —B(OH)₂ or a protected derivative thereof, for example a 4,4,5,5-tetramethyl-1,3,2-dioxaborolan-2-yl group, 9-borabicyclo[3.3.1]-nonane (9-BBN), —Sn(alkyl)₃ (e.g. —SnMe₃ or —SnBu₃), or a similar group known to the skilled person, and A¹, A², A³ and A⁴ are as hereinbefore defined. The skilled person will also appreciate that L^{x} and L^{y} should be mutually compatible, and may also be interchanged), for example in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-biphenyl, 2,2′-bis(diphenylphosphino)-1,1′-bi-naphthyl, 1,1′-bis(diphenylphosphinoferrocene), 1,3-bis(diphenyl-phosphino)propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. The reaction may also be carried out for example at room temperature or above (e.g. at a high temperature such as the reflux temperature of the solvent system) or using microwave irradiation.

Compounds of formula III (or derivatives thereof) in which L³ represents hydroxy:
(A) and R² represents H or C₁₋₆ alkyl optionally substituted by one or more halo atoms, may be prepared by reaction of a compound of formula VIII,

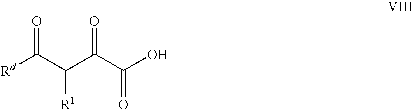

or an enol ether equivalent (e.g. a methyl enol ether or a silyl (e.g. trimethylsilyl) enol ether), or an O-protected (e.g. at the carboxylic acid) derivative thereof, wherein R^{d} and R¹ are as hereinbefore defined, with hydrazine (or a hydrate or derivative (e.g. benzylhydrazine) thereof), for example under conditions such as those described hereinbefore in respect of preparation of compounds of formula I (process (ix) above);
(B) and either one of R¹ or R² represents halo and the other represents H or optionally substituted C₁₋₆ alkyl or both R¹ and R² represent halo, may be prepared by reaction of a corresponding compound of formula III in which R¹ and R² both represent H or one of R¹ or R² represents H and the other represents optionally substituted C₁₋₆ alkyl or halo, with an electrophile that provides a source of halogen atoms, such as one described hereinbefore in respect of process step (i)(b) (for example, chlorine gas), under reaction conditions known to those skilled in the art such as in the presence of a suitable solvent (e.g. water). Thus, relevant 4-halo, 5-halo or 4,5-dihalo substituted pyrazoles may be prepared in such a manner;
(C) and one of R¹ or R² represents fluoro and the other represents H may be prepared from 4-nitropyrazole-3-carboxylic acid or 5-nitropyrazole-3-carboxylic acid (as appropriate) employing an appropriate reagent for the conversion of the nitro group to a fluoro group (such as sodium fluoride, potassium fluoride, tetramethylammonium fluoride or tetrabutylammonium fluoride) under conditions known to those skilled in the art;
(D) and one of R¹ or R² represents halo and the other represents H, may be prepared by reaction of a compound corresponding to a compound of formula III but in which one of R¹ or R² represents amino and the other represents H (as appropriate) followed by conversion of the amino group to a diazonium salt (employing reagents and conditions known to those skilled in the art, e.g. NaNO₂ and HCl at 5° C.) and then the addition of an appropriate nucleophile for the conversion to a halo group. Suitable nucleophiles include potassium, sodium or copper halides. Alternatively, for the introduction of the fluoro group, the appropriate diazonium salt may be treated with a compound that provides a source of fluoroborate (e.g. tetrafluoroborate) salts, for example by introducing a cold aqueous solution of NaBF₄, HBF₄ or NH₄BF₄, so forming the appropriate diazonium fluoroborate (e.g. diazonium tetraflouorborate), which may then be heated;
(E) and R¹ represents halo (e.g. F or Cl) or C₁₋₆ alkyl optionally substituted by one or more halo atoms may be prepared from corresponding compounds of formula III in which R¹ represents H, for example in accordance with a procedure described in R. Storer et al., Nucleosides & Nucleotides 18, 203 (1999). The appropriate reagents that may be employed for the introduction of the halo or optionally substituted C₁₋₆ alkyl group are described hereinbefore in respect of preparation of compounds of formula I (process step (i) above);
(F) and R¹ and R² independently represent perfluoro-C₁₋₆ alkyl or, preferably, H or halo (e.g. chloro) may alternatively be prepared by oxidation of a compound of formula IX,

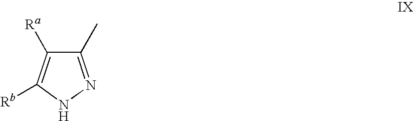

wherein R^{a} and R^{b} independently represent perfluoro-C₁₋₆ alkyl or, preferably, H or halo (e.g. chloro), under oxidation conditions known to those skilled in the art, for example mild or strong (e.g. employing an aqueous solution of potassium permanganate and heating at reflux) oxidation conditions as appropriate;
(G) and R² is as hereinbefore defined (e.g. hydrogen or halo) may be prepared by reaction of a compound of formula X,

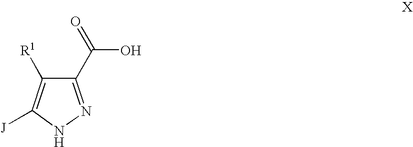

or a N-protected and/or O-protected (e.g. ester) derivative thereof, wherein J and R¹ are as hereinbefore defined. For compounds of formula III in which R² represents halo, reaction may be with a suitable halogenating reagent such as cesium fluoroxysulfate (in the case of a fluorinating reagent) or one described hereinbefore in respect of process step (i)(b), optionally in the presence of a suitable solvent (e.g. hexane, diethyl ether, tetrahydrofuran or 1,4-dioxane or mixtures thereof) under conditions known to those skilled in the art. For compounds of formula III in which R² represents H, reaction may be with reagents and under conditions such as those hereinbefore described in respect of preparation of compounds of formula I (process step (vi));
(H) and R¹ and R² are as hereinbefore defined may be prepared by oxidation of a compound of formula XI,

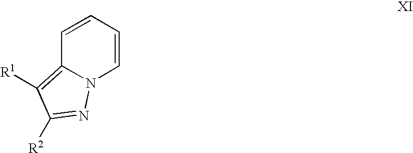

wherein R¹ and R² are as hereinbefore defined, under oxidation conditions known to those skilled in the art, such as those described hereinbefore in respect of preparation of compounds of formula III (i.e. from a compound of formula IX; process step (F) above) above;
(I) and R² represents H and R¹ is as hereinbefore defined (and preferably represents H or C₁₋₆ alkyl optionally substituted as hereinbefore defined) may be prepared by reaction of a compound of formula XII,

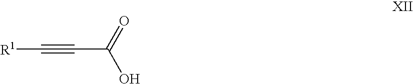

or a protected derivative (e.g. an ester, such as a C₁₋₆ (e.g. ethyl) ester) thereof, wherein R¹ is as hereinbefore defined (and preferably represents H or C₁₋₆ alkyl optionally substituted as hereinbefore defined), with diazomethane, or a protected derivative thereof (e.g. trimethylsilyldiazomethane), for example under conditions known to those skilled in the art (such as in the presence of a suitable solvent (e.g. diethyl ether) and/or at low temperatures (e.g. 0° C. to room temperature)); or
(J) may be prepared by reaction of a corresponding compound of formula XIII,

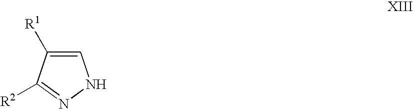

(or a protected derivative thereof), with a suitable base such as one described hereinbefore in process step (i) above, e.g. a lithiation step, followed by reaction with an electrophile that is a source of CO₂ (e.g. CO₂ gas), followed by the addition of a suitable proton source (e.g. HCl), or a compound of formula XIV

L^{1c}C(O)OR^{f} XIV
wherein R^{f} represents C₁₋₆ alkyl and L^{1c} represents a suitable leaving group such as halo (e.g. iodo, bromo or chloro) so forming, for example, methyl or ethyl chloroformate, or the like. The skilled person will therefore appreciate that the pyrazole nitrogen may need to be protected, and, if necessary, subsequently deprotected.

Compounds of formula IV, may be prepared by reduction of a compound of formula XV,

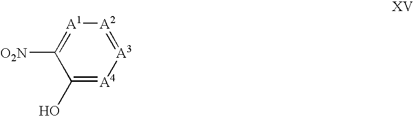

or, a compound of formula XVI,

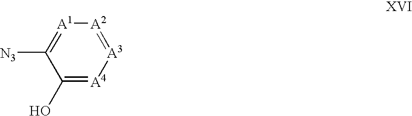

wherein, in both cases, A¹, A², A³ and A⁴ are as hereinbefore defined, for example reduction in the presence of sodium dithionite (Na₂S₂O₄), tin(II) chloride, iron or zinc in the presence of an acid solution, or reduction under hydrogenation conditions in the presence of a catalyst (e.g. palladium or platinum, or the like, on carbon), with a source of hydrogen (e.g. hydrogen gas or nascent hydrogen (e.g. from ammonium formate)), optionally in the presence of a solvent (such as an alcoholic solvent (e.g. methanol) or ethyl acetate).

Compounds of formula V (or protected derivatives thereof) may be prepared by reaction of:
(a) a compound of formula XIII as hereinbefore defined, with a suitable base, such as one hereinbefore described in process step (i) above, e.g. a lithiation step, followed by reaction with a compound of formula XVII,

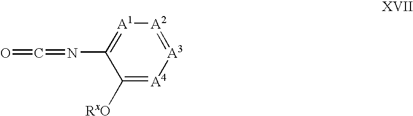

wherein R^{x} represents a hydroxy-protecting group (for example, a standard protecting group such as benzyl) and A¹, A², A³ and A⁴ are as hereinbefore defined, followed by quenching with a suitable proton source (e.g. water or sat., aq. NH₄Cl solution). This reaction may be performed under similar conditions to those described above in respect of preparation of compounds of formula I (process step (i)). The skilled person will therefore appreciate that the pyrazole nitrogen may need to be protected, and, if necessary, subsequently deprotected;
(b) either a compound of formula III in which L³ represents hydroxy, or a N-protected and/or O-protected (e.g. ester) derivative thereof, wherein R¹ and R² are as hereinbefore defined, or a compound of formula XVIII,

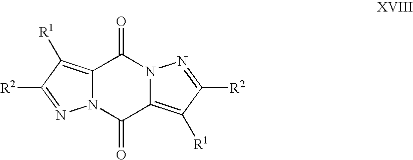

wherein R¹ and R² are as hereinbefore defined, with a compound of formula IV (or a protected derivative, e.g. a —OH protected derivative, thereof) as hereinbefore defined, for example under coupling conditions for example at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]-undec-7-ene, sodium hydroxide, N-ethyldiisopropylamine, N-(methylpolystyrene)-4-(methylamino)pyridine, butyllithium (e.g. n-, s- or t-butyllithium) or mixtures thereof), an appropriate solvent (e.g. tetrahydrofuran, pyridine, toluene, dichloromethane, chloroform, acetonitrile, dimethylformamide, dimethylsulfoxide, water or triethylamine) and a suitable coupling agent (e.g. 1,1′-carbonyldiimidazole, N,N′-dicyclohexylcarbodiimide, 1-(3-dimethylamino-propyl)-3-ethylcarbodiimide (or hydrochloride thereof), N,N′-disuccinimidyl carbonate, benzotriazol-1-yloxytris(dimethylamino) phosphonium hexafluorophosphate, 2-(1H-benzotriazol-1-yl)-1,1,3,3-tetramethyluronium hexafluorophosphate, benzotriazol-1-yloxytrispyrrolidinophosphonium hexafluorophosphate, bromotrispyrrolidinophosphonium hexafluorophosphate, 2-(1H-benzotriazol-1-yl)-1,1,3,3-tetramethyluronium tetrafluorocarbonate, 1-cyclohexylcarbodiimide-3-propyloxymethyl polystyrene, O-(7-azabenzotriazol-1-yl)-N,N,N′,N′-tetramethyluronium hexafluorophosphate or 0-benzotriazol-1-yl-N,N,N′,N′-tetramethyluronium tetrafluoroborate). The skilled person will appreciate that this may also result in the synthesis of compounds of formula VI. Alternatively, for the reaction with a compound of formula III, such compounds may first be activated by treatment with a suitable reagent (e.g. oxalyl chloride, thionyl chloride, etc) optionally in the presence of an appropriate solvent (e.g. dichloromethane, dimethylformamide, THF, toluene or benzene) and a suitable catalyst (e.g. DMF), resulting in the formation of the respective acyl chloride. This activated intermediate may then be reacted with a compound of formula IV under standard conditions, such as those described above. The skilled person will appreciate that when compounds of formula IV are liquid at the reaction temperature, they may serve as both solvent and reactant in this reaction. Alternative methods of performing this step include reaction of an O-protected derivative (e.g. an ethyl ester) of a compound of formula III with a compound of formula IV, which latter compound may first be treated with an appropriate reagent (e.g. trimethylaluminium), for example in an inert atmosphere and in the presence of a suitable solvent (e.g. dichloromethane); or
(c) reaction of a compound of formula XIX,

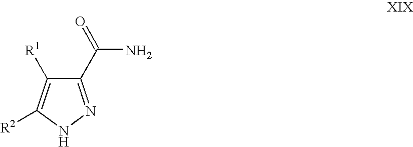

or a N-protected (e.g. at the pyrazole nitrogen) derivative thereof, wherein R¹ and R² are as hereinbefore defined, with a compound of formula XX,

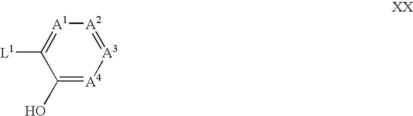

or a protected (e.g. at the requisite —OH group) derivative thereof (so forming, for example, a benzyl-protected compound of formula XX), wherein L¹ represents a suitable leaving group, such as halo (e.g. chloro, bromo and iodo), —OSO₂CF₃, —B(OH)₂, —Sn(R^{z})₃ (wherein R^{z} is as hereinbefore defined), —Pb(OC(O)CH₃)₃, —Bi(W)₂, —Bi(W)₂(OC(O)CH₃)₂, —Bi(W)₂(OC(O)CF₃)₂ or —I(W)(BF₄), and W represents an aryl or heteroaryl group, both of which are optionally substituted by one or more groups selected from X¹ as hereinbefore defined (e.g. W represents the Aⁱ to A⁴ containing phenyl ring, substituted with —OH, of the compound of formula V as hereinbefore defined), and A¹, A², A³ and A⁴ are as hereinbefore defined, for example in the presence of a catalyst containing, preferably, Pd or Cu, and a base, such as potassium or sodium hydroxide, potassium carbonate, potassium tert-butoxide and lithium N,N-diisopropylamide. Catalysts that may be mentioned include Pd₂(dba)₃ (tris(dibenzylideneacetone)dipalladium(0)), bases that may be mentioned include cesium carbonate, ligands that may be mentioned include 2,2′-bis(diphenylphosphino)-1,1′-binaphthyl and solvents that may be employed include toluene. Such reactions may be performed at elevated temperature (e.g. at about 90° C.) under an inert (e.g. argon) atmosphere. The skilled person will appreciate that a protected derivative (e.g. at the —OH group) of a compound of formula XX may be necessary during the above reaction.

Compounds of formula VIIA may be prepared by reaction of a compound of formula XXA,

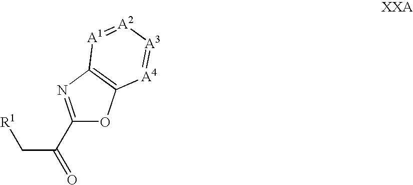

wherein A¹, A², A³, A⁴ and R¹ are as hereinbefore defined, with a compound of formula XXB,

R^{d}—C(O)-L⁴ XXB

wherein L⁴ represents a suitable leaving group, such as halo (e.g. bromo or chloro), or C₁₋₆ alkoxy (e.g. methoxy), and R^{d} is as hereinbefore defined, for example under standard condensation reaction conditions, for example in the presence of a suitable base, an appropriate solvent, and reaction conditions, such as those hereinbefore defined in respect of preparation of compounds of formula I (process step (i)). Preferred bases include metal hydrides (e.g. sodium hydride), or amide bases (e.g. lithium diisopropylamide).

Compounds of formula VIIB may be prepared by reaction of a compound of formula XIII as hereinbefore described, for example under reaction conditions analogous to those hereinbefore described in respect of preparation of compounds of formula I (process step (i)), e.g. in the presence of a suitable base such as one described in that process step (e.g. a lithiation reaction) followed by reaction in the presence of an appropriate electrophilic compound. For instance, for the preparation of a compound of formula VIIB in which LX represents halo, in the presence of an electrophile that provides a source of halogen (e.g. a reagent described in process step (i)(b) above), for the introduction of a sulfonate group, by reaction with a corresponding compound in which there is a hydroxy group present with the appropriate sulfonyl chloride, for the introduction of —Sn(R^{wx})₃, by reaction with the appropriate trialkyl tin chloride (or the like), etc. For the introduction of —B(OH)₂ and —B(OR^{wx})₂, a coupling reaction may be performed from the corresponding halo derivative and the appropriate boronic acid (or derivative thereof) under reaction conditions such as those described in respect of preparation of compounds of formula I (process step (ix)).

Compounds of formula VIIC may be prepared by reaction of a compound of formula XXX, as defined hereinafter, for example under reaction conditions such as those described hereinbefore in respect of preparation of compounds of formula VIIB. Alternatively, compounds of formula VIIC in which L^{y} represents chloro or bromo may be prepared by reaction of a corresponding compound of formula XXC,

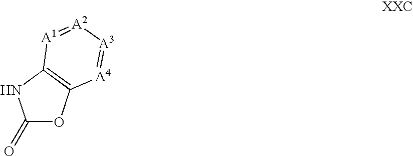

wherein A¹ to A⁴ are as hereinbefore defined, with a suitable reagent that provides a source of bromo or chloro, for example PCl₅, POCl₃ and POBr₃ (or the like).

Compounds of formula IX in which R^{b} represents perfluoro-C₁₋₆ alkyl or halo may be prepared from a corresponding compound of formula IX (or a protected derivative thereof, for example in which the protecting group is a directing metallation group) in which R^{b} represent H, for example under conditions and employing reagents such as those described hereinbefore in respect of preparation of compounds of formula I (process step (i) above).

Alternatively, compounds of formula IX may be prepared by N-dealkylation of a compound of formula XXI,

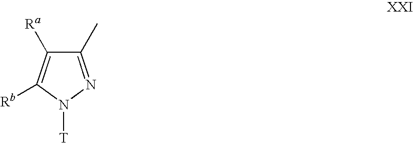

wherein T represents optionally substituted C₁₋₆ alkyl (e.g. methyl) and R^{a} and R^{b} are as hereinbefore defined, under dealkylation conditions known to those skilled in the art, for example by reaction with a suitable reagent (e.g. pyridine hydrochloride) at high temperatures (e.g. 150° C. to 220° C.)). Such a reaction may be carried out in the presence of a suitable solvent, but preferably no further solvent is present.

Alternatively still, compounds of formula IX (and preferably those in which R^{b} represents H or halo) may be prepared from a compound of formula XXII,

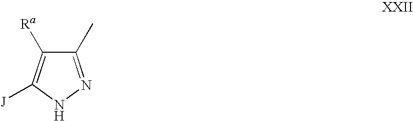

or a N-protected derivative thereof, wherein J and R^{a} are as hereinbefore defined, using reagents and procedures known to those skilled in the art, for example such as those hereinbefore described in respect of preparation of compounds of formula I (process route (vi)) or of formula III (process (G)).

Compounds of formula X (or a N-protected and/or O-protected (e.g. ester) derivative thereof) in which R¹ is as hereinbefore defined and preferably represents H or CF₃, may be prepared by reaction of a compound of formula XXIII,

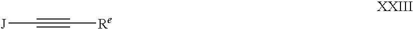

wherein R^{e} represents R¹ as hereinbefore defined and is preferably, H or CF₃, and J is as hereinbefore defined, with a compound of formula XXIV,

N₂—C(H)—C(O)OH XXIV

or a O-protected (e.g. ester) derivative thereof, for example at elevated temperature (e.g. at between 80 and 120° C.) for between 1 and 3 days, optionally in the presence of an inert gas and preferably without the presence of solvent.

Compounds of formula X (or a N-protected and/or O-protected (e.g. ester) derivative thereof) in which R¹ and J are as hereinbefore defined may also be prepared by oxidation of a compound of formula XXII as hereinbefore defined, under oxidation conditions known to those skilled in the art, for example such as those hereinbefore described in respect of preparation of compounds of formula III (process step (F)).

Alternatively, compounds of formula X and XXII (or, where applicable, a N-protected and/or O-protected (e.g. ester) derivative thereof) in which R¹ and J are as hereinbefore defined may be prepared by reaction of a compound of formula XXV,

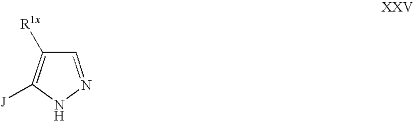

wherein R^{1x} represents R¹ (in the case of preparation of compounds of formula X) or R^{a} (in the case of preparation of compounds of formula XXII) and J, R¹ and R^{a} are as hereinbefore defined, with an appropriate base (or a mixtures of bases), such as those listed in process (i) above, followed by quenching with an appropriate electrophile such as:
(a) for compounds of formula X, a source of CO₂ (e.g. CO₂ gas; which addition is followed by the addition of a suitable proton source (e.g. HCl)), or a compound of formula XIV as hereinbefore defined; or
(b) for compounds of formula XXII, a compound of formula XXVI,

CH₃L^{1d} XXVI
or the like (i.e. another suitable methylating reagent), wherein L^{id} represents a suitable leaving group such as halo (e.g. iodo or bromo) or a sulfonate group (such as —OSO₂CF₃, OSO₂CH₃ and —OSO₂-aryl (e.g. —O-tosyl)).

Compounds of formula XI may be prepared by reaction of 1-aminopyridinium iodide with a compound of formula XXVII,

(R¹)(Cl)C═C(Cl)(R²) XXVII

wherein R¹ and R² are as hereinbefore defined and the geometry of the double bond may be cis or trans, for example under conditions known to those skilled in the art (such as in the presence of a suitable base (e.g. potassium carbonate) and a suitable solvent (e.g. THF)). The skilled person will appreciate that the geometry around the double bond may effect the regioselectivity of the reaction.

Compounds of formula XV in which at least one of —C(R³)═, —C(R⁴)═, —C(R⁵)═ or —C(R⁶)═ is present in which at least one of R³, R⁴, R⁵ and R⁶ represents X¹ in which X¹ represents —S(O)₂N(R^{4h})R^{5h}, may be prepared by reaction of a compound of formula XXVIII,

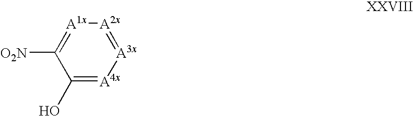

or a protected (e.g. at the requisite —OH group) derivative thereof (so forming, for example, a benzyl-protected —OH group), wherein A^{1x}, A^{2x}, A^{3x} and A^{4x} respectively represent A¹, A², A³ and A⁴ as hereinbefore defined, but in which at least one of —C(R³)═, —C(R⁴)═, —C(R⁵)═ or —C(R⁶)═ is present in which at least one of R³, R⁴, R⁵ and R⁶ represents —S(O)₂Cl, with a compound of formula XXIX,

H₂N(R^{4h})R^{5h} XXIX

wherein R^{4h} and R^{5h} are as hereinbefore defined, for example under conditions known to those skilled in the art (for example, such as those hereinbefore described in respect of preparation of compounds of formula V (process step (b)), e.g. in the presence of a suitable base (e.g. triethylamine) and a suitable solvent (e.g. dichloromethane)). The skilled person will appreciate that a protected derivative (e.g. at the —OH group) of a compound of formula XXVIII may be necessary during either one of the above reaction steps.

Compounds of formula XVIII may be prepared from compounds of formula III (e.g. those in which L³ represents —OH), under dimerising conditions, for example in the presence of thionyl chloride or oxalyl chloride (optionally in the presence of a suitable solvent and catalyst, such as one hereinbefore defined in respect of process step (iii)). Other dimerising reagents include carbodiimides, such as 1,3-dicyclohexylcarbodiimide or 1-(3-dimethylaminopropyl)-3-ethylcarbodiimide (EDCl, or hydrochloride thereof) optionally in the presence of a suitable base (e.g. 4-dimethylaminopyridine).

Compounds of formula XXA may be prepared by reaction of a compound of formula XXX,

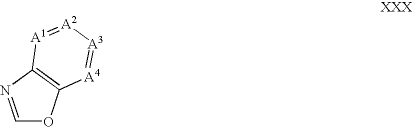

wherein A¹, A², A³ and A⁴ are as hereinbefore defined, with a compound of formula XXXI,

R¹—CH₂—C(O)L⁴ XXXI

wherein R¹ and L⁴ are as hereinbefore defined, in the presence of a suitable base and solvent, and under reaction conditions known to those skilled in the art. For example such conditions may be similar to those described hereinbefore in respect of preparation of compounds of formula I (process step (i) above), e.g. the base may be a metal hydride (e.g. sodium hydride), an amide base (e.g. lithium diisopropylamide) or an organometallic base (such as an organolithium, e.g. n-, s- or t-BuLi), or, alternatively, when L⁴ represents a halo group (e.g. chloro), the reaction may be performed under standard Friedel-Crafts acylation reaction conditions, for instance in the presence of a suitable Lewis acid (e.g. FeCl₃, AlCl₃, BF₃, or the like).

Compounds of formula XXV may be prepared by reaction of a compound of formula XXXII,

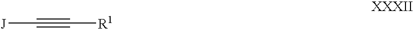

wherein R¹ and J are as defined hereinbefore, with diazomethane under conditions known to those skilled in the art, for example, in accordance with procedures described in T. Hanamoto et al., Chem. Commun., 2041 (2005), e.g. in the presence of a suitable solvent (e.g. hexane, diethyl ether, tetrahydrofuran or 1,4-dioxane or mixtures thereof) and optionally in the presence of an inert gas.

Compounds of formulae II, VI, VII, VIII, XII, XIII, XIV, XV, XVI, XVII, XIX, XX, XXB, XXC, XXI, XXIII, XXIV, XXVI, XXVII, XXVIII, XXIX, XXX, XXXI and XXXII are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

The substituents R¹, R², A¹, A², A³ and A⁴ as hereinbefore defined may be modified one or more times, after or during the processes described above for preparation of compounds of formula I by way of methods that are well known to those skilled in the art. Examples of such methods include substitutions, reductions, oxidations, alkylations, acylations, hydrolyses, esterifications, etherifications, halogenations and nitrations. The precursor groups can be changed to a different such group, or to the groups defined in formula I, at any time during the reaction sequence. In the case where R¹ or R² represents a Cl or F group, such groups may be inter-converted (or converted from another halo group) one or more times, after or during the processes described above for the preparation of compounds of formula I. Appropriate reagents include NiCl₂ (for the conversion to a chloro group). The skilled person may also refer to “Comprehensive Organic Functional Group Transformations” by A. R. Katritzky, O. Meth-Cohn and C. W. Rees, Pergamon Press, 1995 and/or “Comprehensive Organic Transformations” by R. C. Larock, Wiley-VCH, 1999.

Other transformations that may be mentioned include the conversion of a halo group (preferably iodo or bromo) to a cyano or 1-alkynyl group (e.g. by reaction with a compound which is a source of cyano anions (e.g. sodium, potassium, copper (I) or zinc cyanide) or with a 1-alkyne, as appropriate). The latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine). Further, amino groups and hydroxy groups may be introduced in accordance with standard conditions using reagents known to those skilled in the art.

Compounds of the invention may be isolated from their reaction mixtures using conventional techniques.

It will be appreciated by those skilled in the art that, in the processes described above and hereinafter, the functional groups of intermediate compounds may need to be protected by protecting groups. For example the pyrazole nitrogen may need to be protected. Suitable nitrogen-protecting groups include those which form:

(i) carbamate groups (i.e. alkoxy- or aryloxy-carbonyl groups);
(ii) amide groups (e.g. acetyl groups);
(iii) N-alkyl groups (e.g. benzyl or SEM groups);
(iv) N-sulfonyl groups (e.g. N-arylsulfonyl groups);
(v) N-phosphinyl and N-phosphoryl groups (e.g. diarylphosphinyl and diarylphosphoryl groups); or
(vi) N-silyl group (e.g. a N-trimethylsilyl group).

Further, the skilled person will appreciate that, in the case where there are two functional groups protected (e.g. in the case where the carboxylic acid group of the compound of formula III is an ester and the pyrazole nitrogen is protected with a benzenesulfonyl group, then both groups may be deprotected in one step (e.g. a hydrolysis step known to those skilled in the art).

Further protecting groups for the pyrazole nitrogen include a methyl group, which methyl group may be deprotected under standard conditions, such as employing a pyridine hydrochloride salt at elevated temperature, for example using microwave irradiation in a sealed vessel at 200° C.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques.

The type of chemistry involved will dictate the need, and type, of protecting groups as well as the sequence for accomplishing the synthesis.

The use of protecting groups is fully described in “Protective Groups in Organic Synthesis”, 3rd edition, T. W. Greene & P. G. M. Wutz, Wiley-Interscience (1999).

### Medical and Pharmaceutical Uses

Compounds of the invention are useful because they possess pharmacological activity. Such compounds are therefore indicated as pharmaceuticals. According to a further aspect of the invention there is provided a compound of formula I, as hereinbefore defined, or a pharmaceutically-acceptable salt thereof, for use as a pharmaceutical and/or in isolated (i.e. ex vivo) form.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised), may therefore be described as “prodrugs” of compounds of the invention. All prodrugs of compounds of the invention are included within the scope of the invention.

By “prodrug of a compound of the invention”, we include compounds that form a compound of the invention, in an experimentally-detectable amount, within a predetermined time (e.g. about 1 hour), following oral or parenteral administration.

Compounds of the invention are useful because, in particular, they may inhibit the activity of lipoxygenases (and particularly 15-lipoxygenase), i.e. they prevent the action of 15-lipoxygenase or a complex of which the 15-lipoxygenase enzyme forms a part and/or may elicit a 15-lipoxygenase modulating effect, for example as may be demonstrated in the test described below. Compounds of the invention may thus be useful in the treatment of those conditions in which inhibition of a lipoxygenase, and particularly 15-lipoxygenase, is required.

Compounds of the invention are thus expected to be useful in the treatment of inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain and/or fever.

Where a condition has an inflammatory component associated with it, or a condition characterised by inflammation as a symptom, the skilled person will appreciate that compounds of the invention may be useful in the treatment of the inflammatory symptoms and/or the inflammation associated with the condition.

Accordingly, compounds of the invention may be useful in the treatment of asthma, chronic obstructive pulmonary disease (COPD), pulmonary fibrosis, allergic disorders, rhinitis, inflammatory bowel disease, ulcers, inflammatory pain, fever, atherosclerosis, coronary artery disease, vasculitis, pancreatitis, arthritis, osteoarthritis, rheumatoid arthritis, conjunctivitis, iritis, scleritis, uveitis, wound healing, dermatitis, eczema, psoriasis, stroke, diabetes, autoimmune diseases, Alzheimer's disease, multiple sclerosis, sarcoidosis, Hodgkin's disease and other malignancies, and any other disease with an inflammatory component.

Compounds of the invention may also have effects that are not linked to inflammatory mechanisms, such as in the reduction of bone loss in a subject. Conditions that may be mentioned in this regard include osteoporosis, osteoarthritis, Paget's disease and/or periodontal diseases. Compounds of formula I and pharmaceutically acceptable salts thereof may thus also be useful in increasing bone mineral density, as well as the reduction in incidence and/or healing of fractures, in subjects.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, a lipoxygenase (such as 15-lipoxygenase), and/or a method of treatment of a disease in which inhibition of the activity of a lipoxygenase, and particularly 15-lipoxygenase, is desired and/or required (e.g. inflammation), which method comprises administration of a therapeutically effective amount of a compound of formula I as hereinbefore defined but without the proviso, or a pharmaceutically-acceptable salt thereof, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of formula I, as hereinbefore defined, or a pharmaceutically-acceptable salt thereof, in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of formula I, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of inflammation as defined herein (e.g. glucocorticoids or, preferably, NSAIDs, coxibs, corticosteroids, analgesics, inhibitors of 5-lipoxygenase, inhibitors of FLAP (5-lipoxygenase activating protein), and leukotriene receptor antagonists (LTRas), and/or other therapeutic agents that are useful in the treatment of inflammation).

According to a further aspect of the invention, there is provided a combination product comprising:
(A) a compound of formula I, as hereinbefore defined (but, for example, without the proviso), or a pharmaceutically-acceptable salt thereof; and
(B) another therapeutic agent that is useful in the treatment of inflammation,
   wherein each of components (A) and (B) is formulated in admixture with a pharmaceutically-acceptable adjuvant, diluent or carrier.

Such combination products provide for the administration of compound of the invention in conjunction with the other therapeutic agent, and may thus be presented either as separate formulations, wherein at least one of those formulations comprises compound of the invention and at least one comprises the other therapeutic agent, or may be presented (i.e. formulated) as a combined preparation (i.e. presented as a single formulation including compound of the invention and the other therapeutic agent).

Thus, there is further provided:

(1) a pharmaceutical formulation including a compound of formula I, as hereinbefore defined (but, for example, without the proviso), or a pharmaceutically-acceptable salt thereof, another therapeutic agent that is useful in the treatment of inflammation, and a pharmaceutically-acceptable adjuvant, diluent or carrier; and
(2) a kit of parts comprising components:
(a) a pharmaceutical formulation including a compound of formula I, as hereinbefore defined (but, for example, without the proviso), or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of formula I, as hereinbefore defined (but, for example, without the proviso), or a pharmaceutically acceptable salt thereof with the other therapeutic agent that is useful in the treatment of inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective and/or selective inhibitors of lipoxygenases, and particularly 15-lipoxygenase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the stated indications or otherwise.

### Biological Test

The assay employed takes advantage of the ability of lipoxygenases to oxidize polyunsaturated fatty acids, containing a 1,4-cis-pentadiene configuration, to their corresponding hydroperoxy or hydroxyl derivatives. In this particular assay, the lipoxygenase was a purified human 15-lipoxygenase and the fatty acid was arachidonic acid. The assay is performed at room temperature (20-22° C.) and the following are added to each well in a 96-well microtiter plate:

a) 35 μL phosphate buffered saline (PBS) (pH 7.4);
b) inhibitor (i.e. compound) or vehicle (0.5 μl DMSO);
c) 10 μL of a 10× concentrated solution of 15-lipoxygenase in PBS. The plates are incubated for 5 minutes at room temperature;
d) 5 μl of 0.125 mM arachidonic acid in PBS. The plate is then incubated for 10 minutes at room temperature;
e) the enzymatic reaction is terminated by the addition of 100 μl methanol; and
f) the amount of 15-hydroperoxy-eicosatetraenoic acid or 15-hydroxy-eicosatetraenoic acid is measured by reverse phase HPLC.

The invention is illustrated by way of the following examples, in which the following abbreviations may be employed:

aq. aqueous
BuLi n-butyllithium
EtOAc ethyl acetate
EtOH ethanol
MS mass spectrum
NMR nuclear magnetic resonance
PCA pyrazole-3-carboxylic acid
sat. saturated
THF tetrahydrofuran

Chemicals specified in the synthesis of the compounds in the examples were commercially available from, e.g. Sigma-Aldrich Fine Chemicals.

Unless otherwise stated, one or more tautomeric forms of compounds of the examples described hereinafter may be prepared in situ and/or isolated. All tautomeric forms of compounds of the examples described hereinafter should be considered to be disclosed.

### Synthesis of Intermediates

### 5-Chloropyrazole-3-carboxylic acid (I)

The title compound may be prepared from two alternative methods:

### Method A

### (a) 5-Chloro-3-methylpyrazole

A mixture of 5-chloro-1,3-dimethylpyrazole (2.6 mmol) and pyridine hydrochloride (13.1 mmol) in a sealed 5 mL process vial was heated using microwave irradiation for 2 h at 200° C. After cooling to room temperature, EtOAc (15 mL) was added and the mixture was washed with HCl (aq., 2M; 10 mL), NaCl (sat., aq.), dried (MgSO₄) and concentrated to afford the sub-title compound as a white solid (Yield: 210 mg (67%)).

MS (M⁺+H) m/z=117.

¹H-NMR (DMSO-d₆, 400 MHz), δ 12.66 (br s, 1H), 6.03 (m, 1H), 2.19 (s, 3H).

### (b) 5-Chloropyrazole-3-carboxylic acid

A mixture of 5-chloro-3-methylpyrazole (3.6 mmol; see step (a) above), water (6 mL) and tert-butanol (1.2 mL) was heated to 75° C., after which KMnO₄ (1.42 g, 9 mmol) was added. The mixture was stirred at 75° C. overnight and filtered hot. The solids were washed with boiling water. The combined cooled filtrates were extracted with EtOAc, and the combined extracts washed with NaCl (sat., aq.), dried (MgSO₄) and concentrated. The crude solid was recrystallised from EtOAc/hexane/pentane to give the sub-title compound as white crystals (Yield: 350 mg (67%)).

¹H-NMR (DMSO-d₆, 400 MHz), δ 13.65 (br s, 1H), 6.80 (s, 1H).

### Method B

### (a) 1-Benzenesulfonyl-3-methylpyrazole

A mixture of 3-methylpyrazole (5 g, 60.9 mmol), benzenesulfonyl chloride (8.55 mL, 67 mmol) and triethylamine (9.3 mL, 67 mmol) in acetonitrile was heated at reflux for 2 h, allowed to cool and concentrated. EtOAc (300 mL) was added and the solution was filtered and concentrated to provide a solid residue which was crystallised from EtOAc to give the title compound as an off-white powder (Yield: 7.92 g, 58%).

¹H-NMR (DMSO-d₆): δ 8.35 (d, 1H), 7.97-7.94 (m, 2H), 7.78 (tt, 1H), 7.66 (t, 2H), 6.43 (d, 1H), 2.17 (s, 3H).

### (b) 5-Chloro-1-(2-chlorobenzenesulfonyl)-3-methylpyrazole

BuLi (1.6M, 5.9 mL, 9.45 mmol) was added under argon to a solution of 1-benzenesulfonyl-3-methylpyrazole (940 mg, 4.5 mmol; see step (a) above) in THF (50 mL) at −78° C. The mixture was stirred for approximately 30 min before hexachloroethane (3.7 g, 15.8 mmol) was added. After stirring at −78° C. for 18 h, NH₄Cl (sat., aq.; 50 mL) was added and the mixture was allowed to come to room temperature. Water (50 mL) was added, the layers separated, and the aqueous phase extracted with EtOAc (2×100 mL). The combined organic phases were dried (Na₂SO₄) and concentrated. Purification by chromatography (1:4 EtOAc/heptane), followed by recrystallisation from EtOAc/heptane, gave the title compound as white crystals (Yield: 1.1 g, 84%).

¹H-NMR (DMSO-d₆): δ 8.17 (dd, 1H), 7.87-7.67 (m, 4H), 2.15 (s, 3H).

### (c) 5-Chloro-3-methylpyrazole

Sodium ethoxide (2.5M, 16.1 mL, 40.3 mmol) was added to a solution of 5-chloro-1-(2-chlorobenzenesulfonyl)-3-methylpyrazole (6.9 g, 27 mmol; see step (b) above) dissolved in EtOH (50 mL). The solution was stirred for 30 min at room temperature, water (100 mL) was added, the mixture was neutralised using HCl (aq., 2M) and extracted with EtOAc (3×100 mL). Concentration of the combined organic phases resulted in precipitation prior to complete solvent removal. The precipitate was filtered off and the filtrate was concentrated to give the title compound as a brown oil that crystallised on standing (Yield: 1.0 g, 33%) which was used without further purification.

¹H-NMR (DMSO-d₆): δ 12.66 (br s, 1H), 6.03 (d, 1H), 2.20 (s, 3H).

### (d) 5-Chloropyrazole-3-carboxylic acid

A solution of KMnO₄ (3.5 g, 22 mmol) in water (120 mL) was added in portions over a period of 5 h at 70° C. to a solution of 5-chloro-3-methylpyrazole (1.0 g, 8.8 mmol; see step (c) above) in water (50 mL) and tert-butanol (1 mL). The mixture was stirred at 70° C. overnight and filtered through Celite®. The colourless filtrate was concentrated and acidified with HCl (aq., 2M). Filtration gave the title compound as a white powder which was used without further purification. (Yield: 913 mg, 80%).

¹H-NMR (DMSO-d₆): δ 6.80 (s, 1H), 4.40 (br s, 1H).

### 4,5-Dichloropyrazole-3-carboxylic acid (II)

Chlorine gas was bubbled slowly through a stirred solution of 5-chloropyrazole-3-carboxylic acid (Intermediate I, 3.00 g, 20.5 mmol) in water (2.0 L) at room temperature over 3 h. The solution was stirred for 18 h in an open flask and then concentrated in vacuo. The slurry was extracted with ethyl acetate (3×100 mL), the combined extracts were washed with NaCl (sat., aq.; 100 mL) and dried (Na₂SO₄). The solvent was removed in vacuo to give the product as a white powder. Yield 3.20 g (86%).

MS (M⁻−H) m/z=179.

¹H NMR (DMSO-d₆, 400 MHz) δ 14.44 (s, 1H), 14.09 (s, 1H).

¹³C NMR (CD₃OD, 100 MHz) δ 160.0; 139.6; 133.1; 112.4.

### 5-Difluoromethyl-4-chloropyrazole-3-carboxylic acid (III)

### (a) 5-Difluoromethylpyrazole-3-carboxylic acid

KMnO₄ (2.74 g, 9.45 mmol) was added in portions to a mixture of 5-difluoromethyl-3-methylpyrazole (500 mg, 3.78 mmol), t-BuOH (10 mL) and water (100 mL). The mixture was stirred at 75° C. for 18 h. After cooling to room temperature the mixture was filtered and concentrated. HCl (sat., aq.; 2.0 mL) was added and the mixture was extracted with EtOAc (5×20 mL). The combined extracts were washed with NaCl (sat., aq.; 25 mL), dried (Na₂SO₄) and concentrated. The residue was purified using chromatography (reversed phase, RP-18 column and CH₃CN/water (1:2) as eluent. (Yield: 250 mg, 41%).

MS (M⁻−H) m/z=161.

¹H NMR (DMSO-d₆, 400 MHz) δ 14.27 (s, 1H), 13.60 (br. s, 1H), 7.03 (t, 1H), 6.97 (s, 1H).

### (b) 5-Difluoromethyl-4-chloropyrazole-3-carboxylic acid

Chlorine was bubbled slowly through a stirred solution of 5-difluoromethylpyrazole-3-carboxylic acid (100 mg, 0.62 mmol) in water (100 mL) at room temperature over 3 h. The mixture was stirred for 18 h in an open flask and then concentrated. The mixture was extracted with EtOAc (3×20 mL), the combined extracts washed with NaCl (sat., aq.; 25 mL), dried (Na₂SO₄) and concentrated to give the product as a white powder (Yield 106 mg, 87%).

MS (M⁻−H) m/z=195, 197.

### EXAMPLES 1-34

### General Procedure

A mixture of pyrazole-3-carboxylic acid (PCA), 5-chloropyrazole-3-carboxylic acid (intermediate I), 4,5-dichloropyrazole-3-carboxylic acid (intermediate II) or 5-difluoromethyl-4-chloropyrazole-3-carboxylic acid (intermediate III) (1.0 mmol), the relevant aminophenol (1.1 mmol) and polyphosphoric acid (1.0 g) was heated to 160° C. for 18 h. After cooling to 100° C., water (25 mL) and sat. aq. NaHCO₃ (10 mL) were added slowly. After cooling to rt, the mixture was extracted with EtOAc (3×30 mL). The combined organic phases were washed with brine (20 mL) and concentrated in vacuo. The residue was crystallized from EtOAc:heptane to give the desired product.

TABLE 1
Examples (Ex.) 1 to 34

| | | Prepared from | | |
|---|---|---|---|---|
| | | | PCA or Intermediate | |
| Ex. | Name | Aminophenol | (I-III) | Yield % |
| 1 | 2-(Pyrazol-3-yl)- | 2-Aminophenol | PCA | 24 |
| | benzoxazole | | | |
| 2 | 2-(Pyrazol-3-yl)oxazolo[4,5- | 2-Aminopyridin-3-ol | PCA | 2 |
| | b]pyridine | | | |
| 3 | 6-Chloro-2-(pyrazol-3- | 2-Amino-5- | PCA | 15 |
| | yl)benzoxazole | chlorophenol | | |
| 4 | 5-Chloro-2-(pyrazol-3- | 2-Amino-4- | PCA | 23 |
| | yl)benzoxazole | chlorophenol | | |
| 5 | 4-Methyl-2-(pyrazol-3- | 2-Amino-3- | PCA | 36 |
| | yl)benzoxazole | methylphenol | | |
| 6 | 5,7-Dichloro-2-(pyrazol-3- | 2-Amino-4,6- | PCA | 21 |
| | yl)benzoxazole | dichlorophenol | | |
| 7 | 2-(Pyrazol-3-yl)benzoxazol- | 4-Aminobenzene- | PCA | 45 |
| | 6-ol | 1,3-diol | | |
| | | hydrochloride | | |
| 8 | 5-Fluoro-2-(pyrazol-3- | 2-Amino-4- | PCA | 34 |
| | yl)benzoxazole | fluorophenol | | |
| 9 | 5,7-Dichloro-6-methyl-2- | 6-Amino-2,4- | PCA | 34 |
| | (pyrazol-3-yl)benzoxazole | dichloro-3- | | |
| | | methylphenol | | |
| 10 | 6-Fluoro-2-(pyrazol-3- | 2-Amino-5- | PCA | 48 |
| | yl)benzoxazole | fluorophenol | | |
| 11 | 2-(5-Chloropyrazol-3- | 2-Aminophenol | I | 19 |
| | yl)benzoxazole | | | |
| 12 | 2-(4,5-Dichloropyrazol-3- | 2-Aminophenol | II | 28 |
| | yl)benzoxazole | | | |
| 13 | 2-(5-Chloropyrazol-3-yl)-5- | 2-Amino-4- | I | 19 |
| | fluorobenzoxazole | fluorophenol | | |
| 14 | 2-(4,5-Dichloropyrazol-3- | 2-Amino-4- | II | 20 |
| | yl)-5-fluorobenzoxazole | fluorophenol | | |
| 15 | 2-(5-Chloropyrazol-3-yl)-7- | 2-Amino-6- | I | 31 |
| | fluorobenzoxazole | fluorophenol | | |
| 16 | 2-(4,5-Dichloropyrazol-3- | 2-Amino-6- | II | 36 |
| | yl)-7-fluorobenzoxazole | fluorophenol | | |
| 17 | 2-(5-Chloropyrazol-3-yl)-4- | 2-Amino-3- | I | 35 |
| | fluorobenzoxazole | fluorophenol | | |
| 18 | 2-(4,5-Dichloropyrazol-3- | 2-Amino-3- | II | 24 |
| | yl)-4-fluorobenzoxazole | fluorophenol | | |
| 19 | 2-(5-Chloropyrazol-3-yl)-6- | 2-Amino-5- | I | 16 |
| | fluorobenzoxazole | fluorophenol | | |
| 20 | 2-(4,5-Dichloropyrazol-3- | 2-Amino-5- | II | 30 |
| | yl)-6-fluorobenzoxazole | fluorophenol | | |
| 21 | 6-Chloro-2-(5- | 2-Amino-5- | I | 3 |
| | chloropyrazol-3- | chlorophenol | | |
| | yl)benzoxazole | | | |
| 22 | 6-Chloro-2-(4,5- | 2-Amino-5- | II | 7 |
| | dichloropyrazol-3- | chlorophenol | | |
| | yl)benzoxazole | | | |
| 23 | 5-Chloro-2-(4,5- | 2-Amino-4- | II | 4 |
| | dichloropyrazol-3- | chlorophenol | | |
| | yl)benzoxazole | | | |
| 24 | 2-(4,5-Dichloropyrazol-3- | 2-Amino-3,5- | II | 14 |
| | yl)-4,6-difluorobenzoxazole | difluorophenol | | |
| 25 | 2-(5-Chloropyrazol-3-yl)- | 2-Amino-3,5- | I | 18 |
| | 4,6-difluorobenzoxazole | difluorophenol | | |
| 26 | 5-Chloro-2-(5-chloro- | 2-Amino-4- | I | 24 |
| | pyrazol-3-yl)benzoxazole | chlorophenol | | |
| 27 | 2-(4,5-Dichloropyrazol-3- | 2-Amino-4-(tri- | II | 27 |
| | yl)-5-(trifluoromethoxy)- | fluoromethoxy)- | | |
| | benzoxazole | phenol | | |
| 28 | 2-(5-Chloropyrazol-3-yl)-5- | 2-Amino-4- | I | 31 |
| | (trifluoromethoxy)benzoxazole | (trifluoromethoxy)- | | |
| | | phenol | | |
| 29 | 2-(4,5-Dichloropyrazol-3- | 2-Aminopyridin-3-ol | II | 45 |
| | yl)oxazolo[4,5-b]pyridine | | | |
| 30 | 2-(5-Chloropyrazol-3- | 2-Aminopyridin-3-ol | I | 21 |
| | yl)oxazolo[4,5-b]pyridine | | | |
| 31 | 6-Chloro-2-(4,5-dichloro- | 3-Amino-5-chloro- | II | 4 |
| | pyrazol-3-yl)oxazolo[5,4- | pyridin-2-ol | | |
| | b]pyridine | | | |
| 32 | 2-(4,5-Dichloropyrazol-3- | 3-Aminopyridin-2-ol | II | 4 |
| | yl)oxazolo[5,4-b]pyridine | | | |
| 33 | 2-(4,5-Dichloropyrazol-3- | 3-Amino-5-(tri- | II | 25 |
| | yl)-6-(trifluoromethyl)- | fluoromethyl)- | | |
| | oxazolo[5,4-b]pyridine | pyridin-2-ol | | |
| 34 | 6-Chloro-2-[4-chloro-5- | 3-Amino-5- | III | 45 |
| | (difluoromethyl)pyrazol-3- | chloropyridin-2-ol | | |
| | yl]oxazolo[5,4-b]pyridine | | | |

TABLE 2
Physical properties of the Examples 1-34

| | | MS | |
|---|---|---|---|
| | | (M⁻ − H), | |
| Ex. | M.W. | m/z | ¹H NMR (DMSO-d₆, 400 MHz), δ |
| 1 | 185.18 | 184 | 13.6 (br. s, 1H), 7.98 (br. s, 1H), 7.88-7.70 (m, 2H), |
| | | | 7.43-7.40 (m, 2H), 7.00 (d, 1H) |
| 2 | 186.17 | 185 | 10.17 (br. s, 1H), 7.94 (dd, 1H), 7.63 (d, 1H), 7.41 (dd, 1H), |
| | | | 7.15 (dd, 1H), 6.95 (d, 1H) |
| 3 | 219.63 | 218 | 13.63 (s, 1H), 7.99-7.97 (m, 2H), 7.76 (d, 1H), 7.43 (d, |
| | | | 1H), 6.98 (s, 1H) |
| 4 | 219.63 | 218 | 8.01 (d, 1H), 7.87 (d, 1H), 7.81 (d, 1H), 7.45 (dd, 1H), |
| | | | 7.01 (d, 1H) |
| 5 | 199.21 | 198 | 7.96 (br. s, 1H), 7.55 (d, 1H), 7.28 (dd, 1H), 7.18 (d, |
| | | | 1H), 6.99 (d, 1H), 2.56 (s, 3H) |
| 6 | 254.07 | 252 | 8.01 (br. s, 1H), 7.89 (d, 1H), 7.67 (d, 1H), 7.02 (br. s, |
| | | | 1H) |
| 7 | 201.18 | 200 | 9.81 (s, 1H), 7.92 (s, 1H), 7.52 (d, 1H), 7.07 (d, 1H), |
| | | | 6.89 (d, 1H), 6.84 (dd, 1H) |
| 8 | 203.17 | 202 | 13.67 (br. s, 1H), 7.99 (d, 1H), 7.81 (dd, 1H), 7.67 (dd, |
| | | | 1H), 7.29 (ddd, 1H), 7.01 (d, 1H) |
| 9 | 268.10 | 266 | 13.72 (s, 1H), 8.03 (s, 1H), 7.92 (s, 1H), 7.02 (s, 1H), |
| | | | 2.53 (s, 3H) |
| 10 | 203.17 | 202 | 13.63 (br. s, 1H), 8.00 (s, 1H), 7.82-7.76 (m, 2H), |
| | | | 7.28 (ddd, 1H), 6.99 (s, 1H) |
| 11 | 219.63 | 218 | 14.51 (s, 1H), 7.92-7.66 (m, 2H), 7.56-7.28 (m, 2H), |
| | | | 7.09 (s, 1H). |
| 12 | 254.07 | 252 | 14.92 (s, 1H), 7.90-7.72 (m, 2H), 7.52-7.39 (m, 2H) |
| 13 | 237.62 | 236 | 14.61 (s, 1H), 7.95-7.74 (m, 1H), 7.74-7.58 (m, 1H), |
| | | | 7.39-7.18 (m, 1H), 7.14 (s, 1H) |
| 14 | 272.06 | 270 | 15.95 (s, 1H), 7.89-7.86 (m, 1H), 7.78-7.75 (dd, 1H), |
| | | | 7.39-7.34 (m, 1H) |
| 15 | 237.62 | 236 | 14.61 (s, 1H), 7.62 (d, 1H), 7.44-7.34 (m, 2H), 7.16 (s, |
| | | | 1H) |
| 16 | 272.06 | 270 | 15.04 (s, 1H), 7.73 (dd, 1H), 7.50-7.41 (m, 2H) |
| 17 | 237.62 | 236 | 14.62 (s, 1H), 7.69-7.67 (m, 1H), 7.52-7.47 (m, 1H), |
| | | | 7.35-7.31 (m, 1H), 7.19 (s, 1H) |
| 18 | 272.06 | 270 | 15.05 (s, 1H), 7.73-7.70 (m, 1H), 7.55-7.50 (m, 1H), |
| | | | 7.38-7.33 (m, 1H) |
| 19 | 237.62 | 236 | 14.54 (s, 1H), 7.87-7.80 (m, 2H), 7.36-7.30 (m, 1H), |
| | | | 7.08 (s, 1H) |
| 20 | 272.06 | 270 | 14.93 (s, 1H), 7.93-7.84 (m, 2H), 7.38-7.33 (m, 1H) |
| 21 | 254.07 | 252 | 14.56 (s, 1H), 8.01 (d, 1H), 7.85 (d, 1H), 7.50 (dd, 1H), |
| | | | 7.14 (s, 1H) |
| 22 | 288.52 | 286 | 14.98 (s, 1H), 8.01 (d, 1H), 7.85 (d, 1H), 7.57 (dd, 1H) |
| 23 | 288.52 | 286 | 14.98 (s, 1H), 8.00 (d, 1H), 7.89 (d, 1H), 7.54 (dd, 1H) |
| 24 | 290.05 | 288 | 15.02 (s, 1H), 7.76 (dd, 1H), 7.43 (ddd, 1H) |
| 25 | 255.61 | 256¹ | 14.65 (s, 1H), 7.71 (dd, 1H), 7.40 (td, 1H), 7.13 (s, 1H) |
| 26 | 254.07 | 252 | 14.60 (s, 1H), 7.93 (s, 1H), 7.80 (d, 1H), 7.49 (d, 1H), |
| | | | 7.12 (s, 1H) |
| 27 | 338.07 | 336 | 15.05 (s, 1H), 7.99-7.96 (m, 2H), 7.53 (dd, 1H) |
| 28 | 303.62 | 302 | 14.66 (s, 1H), 7.94 (d, 1H), 7.91 (s, 1H), 7.49 (d, 1H), |
| | | | 7.17 (s, 1H) |
| 29 | 255.06 | 253 | 15.12 (s, 1H), 8.62 (dd, 1H), 8.32 (dd, 1H), 7.54 (dd, |
| | | | 1H) |
| 30 | 220.62 | 219 | 14.75 (s, 1H), 8.59 (d, 1H), 8.27 (d, 1H), 7.52 (dd, 1H), |
| | | | 7.24 (s, 1H) |
| 31 | 289.51 | 287 | 12.25 (s, 1H), 8.37 (d, 1H), 7.73 (d, 1H) |
| 32 | 255.06 | 253 | 15.07 (s, 1H), 8.46 (dd, 1H), 8.37 (dd, 1H), 7.57 (dd, |
| | | | 1H) |
| 33 | 323.06 | 321 | 15.20 (s, 1H), 8.91-8.87 (m, 2H) |
| 34 | 305.07 | 303 | 12.21 (s, 1H), 8.38 (d, 1H), 7.74 (d, 1H), 7.37 (t, 1H) |

¹M⁺ + H

### EXAMPLE 35

Title compounds of the Examples were tested in the biological test described above and were found to exhibit an IC₅₀ of 10 μM or below. For example, the following representative compounds of the examples exhibited the following IC₅₀ values:

### Example 1: 224 nM

### Example 4: 443 nM

### Example 11: 33 nM

### Example 15: 41 nM

### Example 17: 49 nM

### Example 18: 35 nM

### Example 32: 127 nM

Example 33: 85 nM

1. A compound of formula I, 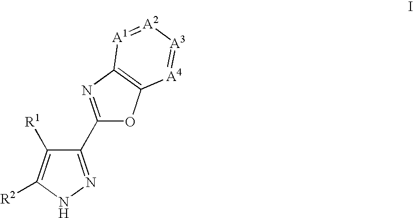
wherein,
R¹ and R² each independently represent H, halo, C₁₋₆ alkyl or —O—C₁₋₆ alkyl, which latter two groups are optionally substituted by one or more halo atoms;
A¹, A², A³ and A⁴ each independently represent —C(R³)═, —C(R⁴)═, —C(R⁵)═ or —C(R⁶)═, or, each of these may alternatively and independently represent —N═;
R³, R⁴, R⁵ and R⁶ each independently represent hydrogen or X¹;
X¹ represents halo, —R^{3a}, —CN, —C(O)R^{3b}, —C(O)OR³ⁿ, —C(O)N(R^{4a})R^{5a}, —N(R^{4b})R^{5b}, —N(R^{3d})C(O)R⁴ⁿ, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR³P, —S(O)₂N(R^{4b})R^{5b}, —S(O)₂OH, —P(O)(OR⁴ⁱ)(OR⁵ⁱ) or —C(O)N(R^{3q})S(O)₂R^{3r};
m represents 0, 1 or 2;
R^{3a} represents C₁₋₆ alkyl optionally substituted by one or more substituents selected from halo, —N(R^{6a})R^{6b}, —N₃, ═O or —OR^{6c};
R^{3b} to R^{3h}, R^{3k}, R³ⁿ, R^{3q}, R^{4a} to R^{4h}, R^{5a}, R^{5b}, R^{5d} and R^{5f} to R^{5h} each independently represent H or C₁₋₆ alkyl optionally substituted by one or more halo atoms or —OR^{6d}; or
any of the pairs R^{4a} and R^{5a}, R^{4b} and R^{5b}, R^{4d} and R^{5d}, R^{4f} and R^{5f}, R^{4g} and R^{5g}, and R^{4h} and R^{5h}, may be linked together to form a 3- to 6-membered ring, which ring optionally contains a further heteroatom in addition to the nitrogen atom to which these substituents are necessarily attached, and which ring is optionally substituted with one or more substituents selected from F, ═O and C₁₋₆ alkyl optionally substituted by one or more fluoro atoms;
R³ⁱ, R^{3j}, R^{3m}, R^{3p} and R^{3r} each independently represent C₁₋₆ alkyl optionally substituted by one or more substituents selected from B¹;
R⁴ⁱ and R⁵ⁱ each independently represent H or C₁₋₆ alkyl optionally substituted by one or more substituents selected from B²;
R^{6a}, R^{6b}, R^{6c} and R^{6d} each independently represent H or C₁₋₆ alkyl optionally substituted by one or more substituents selected from B³; or
R^{6a} and R^{6b} may be linked together to form a 3- to 6-membered ring, which ring optionally contains a further heteroatom in addition to the nitrogen atom to which these substituents are necessarily attached, and which ring is optionally substituted by ═O or C₁₋₆ alkyl optionally substituted by one or more fluoro atoms;
B¹, B² and B³ each independently represent F, Cl, —OCH₃, —OCH₂CH₃, —OCHF₂, —OCH₂CF₃, —OCF₃ or —OCF₂CF₃,
or a pharmaceutically-acceptable salt thereof,
provided that:
when R¹ and R² both represent H, A¹, A², A³ and A⁴ respectively represent —C(R³)═, —C(R⁴)═, —C(R⁵)═ and —C(R⁶)═, and R³ and R⁵ represent H, then both R⁴ and R⁶ do not represent t-butyl.

2. The compound according to claim 1, wherein X¹ represents —C(O)N(R^{4a})R^{5a}, —N(R^{4b})R^{5b}, —N(H)C(O)R^{4c}, —S(O)₂CH₃, —S(O)₂CF₃, —S(O)₂N(R^{4h})R^{5h}, —CN, —NO₂, halo, —R^{3a} or —OR^{3h}.

3. The compound according to claim 2, wherein X¹ represents halo, R^{3a} or —OR^{3h}.

4. The compound according to claim 1, wherein R^{3b}, R^{3c}, R^{3h}, R^{4a} to R^{4h}, R^{5a}, R^{5b}, R^{5d} and R^{5f} to R^{5h} each independently represent hydrogen or C₁₋₄ alkyl, or the relevant pairs are linked together to form a pyrrolidinyl, piperidinyl, morpholinyl or a piperazinyl ring.

5. The compound according to claim 1, wherein R^{3d} to R^{3g} each independently represent C₁₋₂ alkyl or hydrogen.

6. The compound according to claim 1, wherein R³ⁱ and R^{3j} each independently represent C₁₋₄ alkyl optionally substituted by one or more F atoms.

7. The compound according to claim 1, wherein R^{3h} represents H or C₁₋₃ alkyl Optionally substituted by one or more halo atoms.

8. The compound according to claim 1, wherein R^{3a} represents C₁₋₃ alkyl optionally substituted by one or more halo atoms.

9. The compound according to claim 1, wherein R¹ and R² each independently represent H, halo or C₁₋₃ alkyl.

10. The compound according to claim 9, wherein R¹ and R² each independently represent H, Cl or methyl optionally substituted by one or more fluoro atoms.

11. The compound according to claim 1, wherein any one of A¹ to A⁴ represents —N═ or none of A¹ to A⁴ represent —N═, and the others represent —C(R³)═, —C(R⁴)═, —C(R⁵)═ or —C(R⁶)═.

12. The compound according to claim 1, wherein R³, R⁴, R⁵ and R⁶ each independently represent trifluoromethyl, H, methyl, fluoro, chloro or hydroxy.

13. A compound according to claim 1, or a pharmaceutically-acceptable salt thereof, for use as a pharmaceutical.

14. A pharmaceutical formulation including a compound according to claim 1, or a pharmaceutically acceptable salt thereof, in admixture with a pharmaceutically acceptable adjuvant, diluent or carrier.

15. A compound according to claim 1 but without the proviso, or a pharmaceutically acceptable salt thereof, for use in the treatment of a disease in which inhibition of the activity of a lipoxygenase is desired or required.

16. Use of a compound according to claim 1 but without the proviso, or a pharmaceutically acceptable salt thereof, for the manufacture of a medicament for the treatment of a disease in which inhibition of the activity of a lipoxygenase is desired and/or required.

17. A compound as claimed in claim 15, or a use as claimed in claim 16, wherein the lipoxygenase is 15-lipoxygenase.

18. The use according to claim 16, wherein the disease is inflammation and/or has an inflammatory component.

19. The use according to claim 18 wherein the inflammatory disease is asthma, chronic obstructive pulmonary disease, pulmonary fibrosis, an allergic disorder, rhinitis, inflammatory bowel disease, an ulcer, pain, inflammatory pain, fever, atherosclerosis, coronary artery disease, vasculitis, pancreatitis, arthritis, osteoarthritis, rheumatoid arthritis, conjunctivitis, iritis, scleritis, uveitis, a wound, dermatitis, eczema, psoriasis, stroke, diabetes, autoimmune diseases, Alzheimer's disease, multiple sclerosis, sarcoidosis, Hodgkin's disease or another malignancy.

20. A method of treatment of a disease in which inhibition of the activity of a lipoxygenase is desired or required, said method comprising administration of a therapeutically effective amount of a compound according to claim 1 but without the proviso, or a pharmaceutically-acceptable salt thereof, to a patient suffering from, or susceptible to, such a condition.

21. A combination product comprising:
(A) a compound according to claim 1 but without the proviso, or a pharmaceutically-acceptable salt thereof; and
(B) another therapeutic agent that is useful in the treatment of inflammation,
wherein each of components (A) and (B) is formulated in admixture with a pharmaceutically-acceptable adjuvant, diluent or carrier.

22. A combination product according to claim 21 which is useful in the treatment of inflammation, and a pharmaceutically-acceptable adjuvant, diluent or carrier.

23. A combination product which comprises a kit of parts comprising components:
(a) a pharmaceutical formulation including a compound according to claim 1 but without the proviso, or a pharmaceutically-acceptable salt thereof, in admixture with a pharmaceutically-acceptable adjuvant, diluent or carrier; and
(b) a pharmaceutical formulation including another therapeutic agent that is useful in the treatment of inflammation in admixture with a pharmaceutically-acceptable adjuvant, diluent or carrier,
which components (a) and (b) are each provided in a form that is suitable for administration in conjunction with the other.

24. A process for the preparation of a compound of formula I as defined in claim 1, which comprises:
(i) for compounds of formula I in which R² represents halo or C₁₋₆ alkyl Optionally substituted by one or more halo atoms), reaction of a corresponding compound of formula I in which R² represents hydrogen, with an appropriate base or a mixture of bases, followed by quenching with:
(a) for compounds of formula I in which R² represents an optionally substituted C₁₋₆ alkyl group, a compound of formula II,
R^{c}L^{1a} II
wherein R^{c} represents C₁₋₆ alkyl optionally substituted by one or more halo atoms, and L^{1a} represents a suitable leaving group, or, for compounds of formula I in which R² represents CF₃, a trifluoromethylating reagent; or
(b) for compounds of formula I in which R² represents halo, an electrophile that provides a source of such atoms;
(ii) for compounds of formula I in which R¹ and/or R² represent C₁₋₆ alkoxy optionally substituted by one or more halo atoms), reaction of a compound corresponding to a compound of formula I but in which in place of the relevant substituents R¹ and/or R², (a) hydroxy group(s) is/are present, with a compound of formula II as defined above, or for the introduction of a methoxy group at R¹ or R² with diazomethane;
(iii) for compounds of formula I in which R² represents CF₃, reaction of a corresponding compound of formula I in which R² represents bromo or iodo with CuCF₃ or a source of CuCF₃;
(iv) reaction of a compound of formula III, 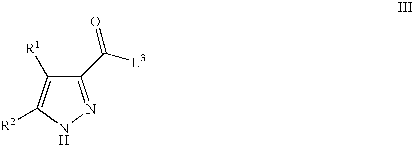
or a protected derivative thereof, wherein R¹ and R² are as defined in claim 1 and L³ represents a suitable leaving group, with a compound of formula IV, 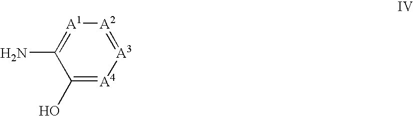
wherein A¹, A², A³ and A⁴ are as defined in claim 1;
(v) intramolecular cyclisation of a compound of formula V, 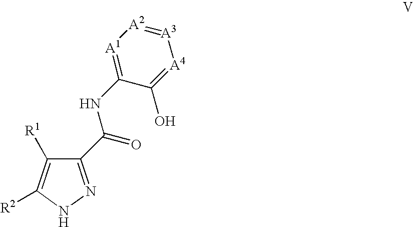
or a compound of formula VI, 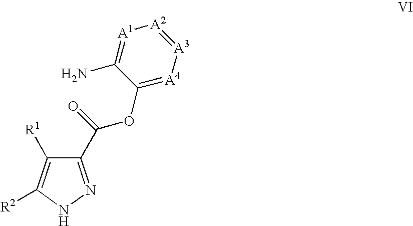
wherein R¹, R², A¹, A², A³ and A⁴ are as defined in claim 1;
(vi) for compounds of formula I in which R² represents hydrogen and R¹ is as defined in claim 1, removal of the group J from a compound of formula VII, 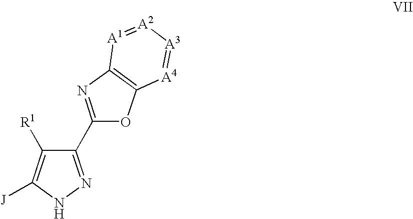
wherein J represents —Si(R^{t})₃ or —Sn(R^{z})₃ wherein each R^{t} independently represents a C₁₋₆ alkyl group or an aryl group and wherein each R^{z} independently represents C₁₋₆ alkyl, and R¹, A¹, A², A³ and A⁴ are as defined in claim 1;
(vii) for compounds of formula I in which one of R¹ or R² represents an optionally substituted C₁₋₆ alkyl group, chloro or fluoro and the other represents H, reaction of a corresponding compound of formula I in which one of R¹ or R² represents bromo or iodo and the other represents H with a suitable organolithium base, followed by quenching with a compound of formula II, as defined above, or a source of chlorine or fluorine atoms;
(viii) for compounds of formula I in which R² represent H or C₁₋₆ alkyl optionally substituted by one or more halo atoms, reaction of a compound of formula VIIA, 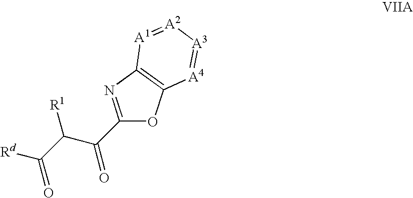
wherein R^{d} represents H or C₁₋₆ alkyl optionally substituted by one or more halo atoms and R¹ is as defined above, with hydrazine or a hydrate or derivative thereof);
(ix) reaction of a compound of formula VIIB, 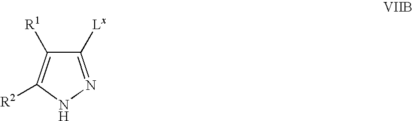
wherein L^{x} represents a suitable leaving group, and R¹ and R² are as defined in claim 1, with a compound of formula VIIC, 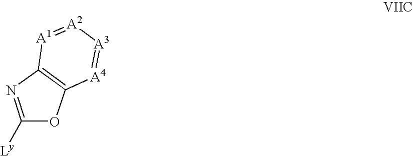
wherein L^{y} represents a suitable leaving group, and A¹, A², A³ and A⁴ are as defined in claim 1.

25. A process for the preparation of a pharmaceutical formulation, the process comprises bringing into association a compound according to claim 1, or a pharmaceutically acceptable salt thereof with a pharmaceutically-acceptable adjuvant, diluent or carrier.

26. A process for the preparation of a combination product, the process comprises bringing into association a compound according to claim 1 but without the proviso, or a pharmaceutically acceptable salt thereof with the other therapeutic agent that is useful in the treatment of inflammation, and at least one pharmaceutically-acceptable adjuvant, diluent or carrier.
